(19) United States
(12) Patent Application Publication
(10) Pub. No.: US 2026/0174860 A1
(43) Pub. Date: Jun. 25, 2026

(54) HYBRID COMPOUNDS OF SCLAREOL AND DOXORUBICIN, THEIR SYNTHESIS AND APPLICATION

(71) Applicant: INSTITUTE FOR BIOLOGICAL RESEARCH "SINISA STANKOVIC" - NATIONAL INSTITUTE OF THE REPUBLIC OF SERBIA, Belgrade (RS)

(72) Inventors: Milica Pesic, Belgrade (RS); Igor Opsenica, Belgrade (RS); Ana Kostic, Smederevo (RS); Natasa Terzic-Jovanovic, New Belgrade (RS)

(21) Appl. No.: 19/129,317

(22) PCT Filed: Oct. 25, 2023
(86) PCT No.: PCT/RS2023/000016

§ 371 Date: May 13, 2025

(51) Int. Cl. A61K 45/06

(52) U.S. Cl. CPC A61K 45/06

(57) ABSTRACT

The invention represents new hybrid compounds of two natural products sclareol and doxorubicin in the form of their conjugates. These compounds are in the form of conjugates, where doxorubicin and sclareol are covalently linked by a linker in a 1:1 molar ratio. The hybrids have shown to possess anticancer properties and are effective in treating resistant cancer cells that have P-glycoprotein membrane transporter, responsible for resistance to doxorubicin. The hybrids have been tested on different types of cell lines, including human glioblastoma, non-small cell lung carcinoma, and colorectal carcinoma. Also, a method for their preparation and their use in medical products or pharmaceutical preparations has been determined. The results of the study include the cytotoxic activity of single, combined, and conjugated compounds in pairs of sensitive and resistant cancer cells, with and without P-glycoprotein expression. The selectivity towards cancer cells was determined by comparing with commercially available normal human lung fibroblast cells. The study also investigated the nanoparticle nature of hybrid compounds, their intracellular localization and toxicity in vivo.

### FIELD OF INVENTION

The present invention relates to the synthesis of novel hybrid compounds (conjugates) of sclareol (SC) and doxorubicin (DOX), and their application in anticancer therapy.

### Technical Problem

The technical problem solved by the present invention is to find new hybrid compounds that exhibit potent anticancer effects against different types of cancer cells, such as human glioblastoma, non-small cell lung carcinoma, and colorectal carcinoma. Additionally, the invention provides a method for producing these hybrid compounds from the appropriate ligands and to use of compounds thus obtained in medicinal products or pharmaceutical preparations/compositions.

### BACKGROUND

The study conducted by Borges GSM, et al. evaluates the effects of the combined administration of SC and DOX in anticancer therapy. The research found that a combination of doxorubicin (DOX) and sclareol (SC) in a molar ratio of 1:1.9 achieves a better synergistic anticancer effect than a molar ratio of 1:7.5 on cancer cell lines in breasts. The use of nanolipid carrier with DOX and SC further improves cytotoxicity in cell lines and animal models compared to free DOX. However, the combination without the nanolipid carrier caused a loss in body weight, behavioral change, and hematological toxicity in animals. On the other hand, the application of the combination with the nanolipid carrier did not cause any side effects. The study recommends the delivery of DOX and SC in a ratio below 1:2 inside nanoparticles for desirable anticancer effects (Life Sci. 2019 Sep. 1; 232:116678. doi: 10.1016/j.lfs.2019.116678. Epub 2019 Jul. 22. PMID: 31344429).

The research carried out by Oliveira M S et. al. demonstrated that the combination of DOX and SC in a suspension of solid lipid nanoparticles increases its cytotoxic effect on cancer cells. The study found that DOX is released from the lipid nanoparticles at pH 7.4, with higher release at lower pH values. However, SC alone packed in lipid nanoparticles did not exhibit any significant anticancer effect (J Nanosci Nanotechnol. 2018 Aug. 1;18 (8): 5609-5616. doi: 10.1166/jnn.2018.15418. PMID: 29458616). Moreover, the research conducted by Perce F and Torchilin V P indicated that SC did not enhance the accumulation of DOX in spheroids of ovarian cancer cell lines that are resistant to DOX. However, it did increase the cytotoxicity of DOX by 4.5 times (Cancer Biol Ther. 2012 October;13 (12): 1205-13. doi: 10.4161/cbt.21353. Epub 2012 Aug. 15. PMID: 22892843).

Finally, a study by Dimas K et. al. revealed that SC significantly boosted the cytotoxic effect of DOX in breast cancer cell lines. When given together, 50 μM SC and 1 μM DOX proved to be highly cytotoxic compared to when SC or DOX were administered alone. In fact, a combination of 50 μM SC and 0.1 μM DOX also significantly reduced the rate of cell growth, showing 76% when DOX administered alone, while only 4% in combination with SC (Biomed Pharmacother. 2006 April;60 (3): 127-33. doi: 10.1016/j.biopha.2006.01.003. Epub 2006 Feb. 21. PMID: 16527443).

The subject invention describes the results of the impact of combined treatments using two different concentrations of SC along with five different concentrations of DOX on eight different human cell lines, as well as the results of the specific effects on different types of carcinomas in comparison to normal non-cancer cells.

There is no existing patent document that describes a conjugates of SC and DOX that are covalently linked used in anticancer therapy. Patent databases searches revealed several patent documents that disclose the use of SC and derivatives thereof in treating of infections caused by microorganisms, as well as in cosmetic preparations, as for example EP1083895A2.

Moreover, as prior art document that described the use of DOX in combination with predominantly cytotoxic agents like paclitaxel, docetaxel, and gemcitabine for the treatment of cancer, is NZ581183A.

Data from the Literature on the Derivatization of SC:

The process of modifying SC through chemical reactions is a well-known technique, as described in the literature. For instance, Rehman et al. demonstrated the synthesis of anticancer SC analogs through chemical transformations in their article “Design and Synthesis of Heck-Coupled Sclareol Analogs: Modulation of BH3 Family Members by SS-12 in Autophagy and Apoptotic Cell Death” published in the Journal of Medicinal Chemistry in April 2015. The authors reported that the resulting derivatives were more effective than SC in terms of their biological activity (J. Med. Chem. 2015 Apr. 23;58 (8): 3432-44. doi: 10.1021/jm501942m. Epub 2015 Apr. 9. PMID: 25825934).

### SUMMARY OF THE INVENTION

The invention provides novel hybrid compounds (conjugates) of SC and DOX, which have been synthesized and obtained with improved selectivity towards cancer cells compared to DOX alone. These conjugates are expected to have less systemic toxicity and cardiotoxicity than DOX, thus addressing its serious limitations. The present invention offers a novel approach to the therapy of different cancer types, including but not limited to non-small cell lung carcinoma, colorectal carcinoma, and glioblastoma, which holds great promise in overcoming resistance to existing treatments and improving patient outcomes.

The present invention presents new hybrid compounds (conjugates) of SC and DOX in treatment of different cancer types, including but not limited to non-small cell lung carcinoma, colorectal carcinoma, and glioblastoma. These malignancies are known to be resistant to available and experimental treatments due to inherent or induced resistance. The blood-brain barrier, which contains P-glycoprotein, is a key obstacle in glioblastoma therapy, as it prevents the penetration of xenobiotics into the brain. The fourth generation of P-glycoprotein inhibitors, represented by natural products, has shown promise in overcoming resistance in these malignancies. Non-small cell lung carcinoma and colorectal carcinoma are common malignancies that affect nearly half of cancer patients. Glioblastoma, on the other hand, is the most common type of brain cancer, and unfortunately, it is also the most resistant to available and experimental treatments.

Resistance can be either inherent or induced, and it is the main obstacle to effective therapy for these three malignancies. Currently, different innovative types of therapy, such as targeted therapy and immunotherapy, are available for non-small cell lung carcinoma and colorectal carcinoma. However, the only chemotherapeutic agent approved for glioblastoma therapy is temozolomide.

Since 2005, not a single new type of chemotherapy has been approved for glioblastoma treatment, which is a significant challenge.

One of the key obstacles in glioblastoma therapy is the blood-brain barrier. This barrier contains P-glycoprotein, a membrane transport protein that does not allow the penetration of foreign substances into the brain. P-glycoprotein is found on the cell membrane of resistant cancer cells, including glioblastoma, non-small cell lung carcinoma, and colorectal carcinoma, where it has a protective role against chemotherapy and radiotherapy. P-glycoprotein also protects cancer cells from innovative targeted therapies.

Natural products, such as bioactive substances isolated from plants and microorganisms, represent the fourth generation of P-glycoprotein inhibitors. Combining natural products-P-glycoprotein inhibitors with chemotherapeutics and innovative therapeutics are expected to solve the problems in the therapy mentioned malignancies such as resistance.

Sclareol (SC) is one such natural product that inhibits the activity of P-glycoprotein.

Doxorubicin (DOX), an anticancer drug which is a P-glycoprotein substrate, is used to treat various types of cancers. However, use thereof has serious limitations, such as the development of resistance, the inability to pass through the blood-brain barrier, and cardiotoxicity.

Hybrid compounds (conjugates) of SC and DOX synthesized and obtained according to the subject invention have better selectivity towards cancer cells than DOX alone. This indicates that the conjugates can have less systemic and cardiotoxicity compared to DOX alone.

The present invention provides a method for synthesis new hybrid compounds CON1 and CON2 of SC and DOX for use in treating of resistant cancers. The hybrid compounds are covalently linked via a linker in a molar ratio of 1:1 to ensure the simultaneous delivery of both chemical entities to cancer cells. The conjugates have been shown to have greater selectivity towards cancer cells and lower resistance than DOX. The present invention also describes the synthesis of new derivatives of SC named ligands LIG1 and LIG2 as precursors for the synthesis of the hybrid compounds.

The resistance of cancer cells expressing P-glycoprotein is lower to hybrid compounds (conjugates CON1 and CON2) of SC and DOX than to DOX alone. This suggests that conjugates can be used in the therapy of resistant cancers, including brain cancers as they can pass through the blood-brain barrier. The results of the subject invention show that the conjugates of a P-glycoprotein inhibitor such as SC and anticancer drug such as DOX are less damaging to normal cells and exhibit lower systemic toxicity compared to DOX alone. The intracellular localization of the conjugates of SC and DOX is in the cytoplasm and nucleus, while the intracellular localization of DOX is in the nucleus, indicating that the conjugates are less damaging to DNA than doxorubicin alone, which is in agreement with the expected lower systemic toxicity of the conjugates.

The subject invention relates to the synthesis of new hybrid compounds (conjugates) in which DOX and SC are covalently linked via a linker in a molar ratio of 1:1, ensuring the simultaneous delivery of both chemical entities to the cancer cells.

The invention also describes the synthesis of new derivatives of SC named ligands LIG1 and LIG2, which represent precursors in the synthesis of hybrid compounds (CON1 and CON2, respectively) of SC and DOX.

The newly synthesized hybrid compounds (CON1 and CON2) exhibit favorable anticancer characteristics that include greater selectivity towards cancer cells compared to the starting compounds SC and DOX, as well as a lower degree of resistance than DOX. These hybrid compounds were found to be effective against different types of cell lines, including human glioblastoma, non-small cell cancer, and colorectal cancer.

While combining SC and DOX as anticancer therapy is known in the literature, the novelty of the present invention is that for the first time the synthesis of hybrid molecules of SC and DOX, in which these structures are linked by a covalent bond is disclosed, as well as their anticancer properties.

Disclosed is a method for producing hybrid compounds that are conjugates of SC and DOX. The method involves a convergent synthesis in three reaction steps. The hybrid compounds are characterized by the covalent linkage of SC and DOX. The hybrid compounds (CON1 and CON2) exhibit an enviable evading of P-glycoprotein activity and contribute to increased accumulation of DOX in cells with increased expression of P-glycoprotein. The hybrid compounds (CON1 and CON2), conjugates of SC and DOX are particularly expected to make a breakthrough in the therapy of glioblastoma. It has been shown that hybrid structures based on natural products and/or drugs perform significantly better pharmacokinetic and pharmacodynamic properties in relation to individual molecules.

The following invention outlines a process for producing hybrid compounds that combines SC and DOX in a convergent synthesis involving three reaction steps. Two new hybrid compounds, CON1 and CON2, conjugates containing different ligand derivatives, were synthesized, namely SC derivatives LIG1 and LIG2, respectively. These conjugates were characterized and their antitumor effects were tested on different human cell lines. 5 The simultaneous administration of single compounds SC and DOX as anticancer therapy has been previously documented in the literature. This has been achieved by combining these two compounds, as well as by delivering them within the same lipid nano-carriers.

The present invention describes, for the first time, the synthesis of hybrid molecules SC and DOX. This means that compounds in which these structures are covalently linked have been created and their in vitro anticancer effects have been tested on different types of cell lines, including human glioblastoma, non-small cell lung carcinoma, and normal human fibroblasts.

The experimental results indicate that the synergistic antitumor effect is better achieved when sclareol and doxorubicin are combined in a ratio closer to 1:1. Accordingly, by merging the two new antitumor compounds through the linker by covalent connection into a hybrid molecule (conjugate), an agent characterized by overcoming obstacles and limitations in the application of individual compounds is achieved.

The obstacles and limitations in the use of the drug are the development of resistance and the cytotoxic effect on normal cells. These obstacles are overcome by the hybridization of SC with DOX, which exhibits an enviable circumvention of P-glycoprotein activity and contributes to increased accumulation of DOX in cells with increased expression of P-glycoprotein.

Moreover, these hybrid compounds, conjugates of SC and DOX, are expected to make a breakthrough in the therapy of glioblastoma. This is because the main obstacle to the application of DOX is the blood-brain barrier which includes the transport pump P-glycoprotein.

Therefore, the conjugates of SC and DOX represent the favorable properties of the antitumor strategy personified in combining these two compounds. It is important to point out that combining two privileged structures into one molecule is in accordance with the latest medical trends in chemistry. It has been shown that hybrid structures based on natural products and/or drugs perform significantly better pharmacokinetic and pharmacodynamic properties in relation to individual molecules.

In the design and development of hybrid molecules, the inventors of this invention selected succinic acid as the linker, i.e., the structural motif that covalently connects sclareol and doxorubicin. It was determined that the conformational flexibility of succinates, which allows for free rotation of the carbon skeleton, facilitates molecule passage through biological membranes and enhances the bioavailability of the hybrids. An amide bond was chosen as the basis for linking sclareol and doxorubicin into a unified structure via the linker due to its high stability under in vivo conditions. Furthermore, the high thermodynamic stability of the amide bond allows for formation thereof under mild reaction conditions with a high yield of the desired products.

The invention primarily provides hybrid compounds of sclareol and doxorubicin, CON1 and CON2, of general formula:

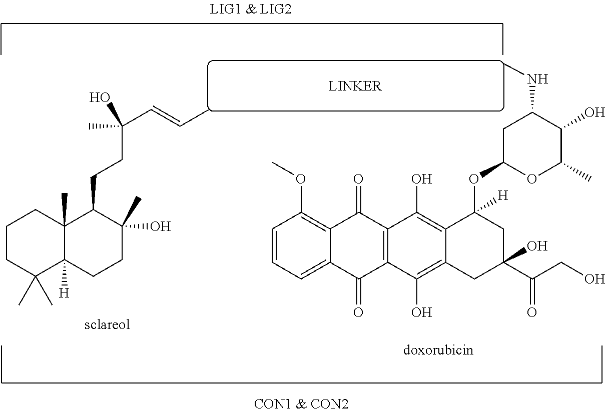

where ligands LIG1 and LIG2 are defined as

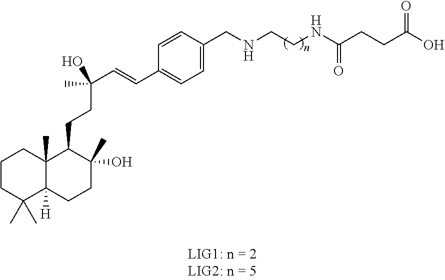

described in the specification below.

More specifically, the invention relates to hybrid compounds CON1 and CON2 of formulas:

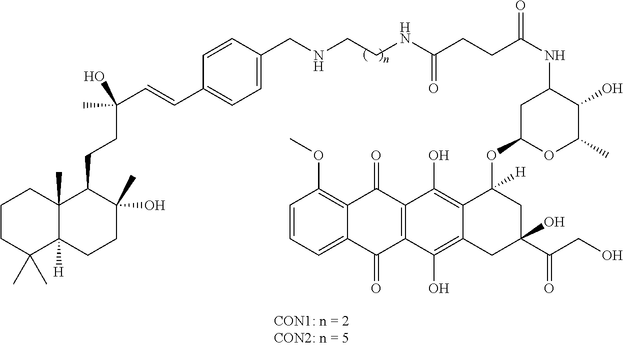

in which doxorubicin and sclareol are covalently linked via the linker in a 1:1 molar ratio, as further described in the specification below.

Furthermore, the invention offers compounds LIG1 and LIG2 of formulas:

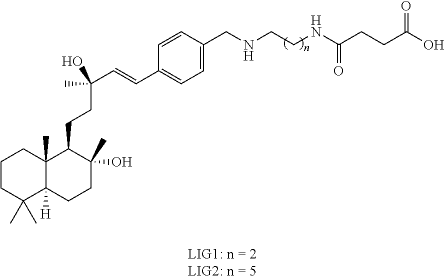

as defined in the following text, which can be particularly useful as precursors in the synthesis of compounds CON1 and CON2.

Additionally, the invention provides hybrid compounds of sclareol and doxorubicin, specifically for use in medical products or pharmaceutical compositions/formulations.

The compounds of the invention, CON1 and CON2, obtained as described here, are defined as:
CON1: N1-((25,3S,4S,6R)-3-hydroxy-2-methyl-6-(((1S,3S)-3,5,12-trihydroxy-3-(2-hydroxyacetyl)-10-methoxy-6,11-dioxo-1,2,3,4,6,11-hexahydrotetracen-1-yl)oxy)tetrahydro-2H-pyran-4-yl)-N4-(3-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)propyl) succinamide and/or pharmaceutically acceptable salt or hydrate or solvate thereof; and
CON2: N1-((2S,3S,4S,6R)-3-hydroxy-2-methyl-6-(((1S,3S)-3,5,12-trihydroxy-3-(2-hydroxyacetyl)-10-methoxy-6,11-dioxo-1,2,3,4,6,11-hexahydrotetracen-1-yl)oxy)tetrahydro-2H-pyran-4-yl)-N4-(6-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)hexyl) succinamide and pharmaceutically acceptable salt and/or hydrate and/or solvate thereof.

Furthermore, compounds LIG1 and LIG2 of the invention, which serve as ligands for covalently binding with doxorubicin, are defined as:
LIG1: 4-((3-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)propyl)amino)-4-oxobutanoic acid, and
LIG2: 4-((6-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)hexyl)amino)-4-oxobutanoic acid.

Furthermore, the present invention encompasses the synthesis of sclareol derivative 1, with the formula: 4-{(1E,3R)-3-hydroxy-5-[(2R,4aS,8aS)-2-hydroxy-2,5,5,8a-tetramethyldecahydronaphthalen-1-yl]-3-methylpent-1-en-1-yl}benzaldehyde, which serves as the starting material for the synthesis of precursors LIG1 and LIG2, and consequently, hybrid compounds CON1 and CON2.

### A Brief Description of Drawings

FIG. 1 depicts inhibition of cell growth (cytotoxicity) in response to increasing concentrations of SC (5, 10, 20, 50 and 100 μM) in different cell lines, including U87 and U87-TxR (sensitive and resistant glioblastoma cell lines), U251 (another glioblastoma cell line), NCI-H460 and NCI-H460/R (sensitive and resistant non-small cell lung carcinoma cell lines), DLD1 and DLD1-TxR (sensitive and resistant colorectal carcinoma cell lines) and MRC-5 (human lung fibroblasts-normal non-cancer cells). The left panel shows a non-linear function of cell growth inhibition, while the right panel shows “IC50” values. IC50 is the concentration that causes 50% cell growth inhibition relative to control untreated cells. These values were obtained by non-linear regression using GraphPad software Prism 8.0.2. The MTT assay was used, and treatment lasted 72 h.

FIG. 2 presents the interaction of SC and DOX at the level of cytotoxicity in different cells. SC (10 and 20 μM) was combined with increasing concentrations of DOX (20, 50, 100, 200 and 500 nM). The co-treatment lasted 72 h, and the MTT assay was used to determine the non-linear regression obtained using GraphPad Prism 8.0.2 software (line graphs). Additionally, the combination indices (CI) for tested co-treatments of SC and DOX were obtained using CalcuSyn software (dot plots). CI values reflect the type of interactions of two combined compounds, where an additive effect is represented by Cl≈1, synergism is represented by Cl<1, and antagonism is represented by Cl≈1.

FIG. 3 illustrates the increased accumulation of DOX in all glioblastoma cell lines, regardless of the expression and activity of P-glycoprotein (which is present in excess only in U87-TxR). The combined treatment with 20 μM and 50 μM SC was found to increase the accumulation of 20 μM DOX, which was analyzed on a flow cytometer after 60 min of combined exposure.

FIG. 4 shows that in resistant cells NCI-H460/R, the accumulation of DOX increased along with the accumulation of rhodamine 123, indicating the inhibition of P-glycoprotein activity in the presence of 50 μM of SC. However, P-glycoprotein-deficient NCI-H460 cells did not show any increase in DOX or rhodamine 123 accumulation. The accumulation of 20 μM DOX was measured on a flow cytometer after 60 min of exposure to the combined treatment of SC and DOX (left panel), while the accumulation of 5 M rhodamine 123 was measured on a flow cytometer after 30 min of exposure to the combined treatment of SC and rhodamine 123 (right panel).

FIG. 5 presents the resistance profile of two conjugates, CON1 and CON2, and DOX on sensitive U87 and resistant U87-TxR. The study used a range of concentrations (2.5, 5, 10, 25, and 50 μM) for CON1 and CON2, and range of concentrations (0.25, 0.5, 1, 2.5, and 5 μM) for DOX and lasted for 72 h, using the MTT assay. GraphPad Prism 8.0.2 software was used to obtain non-linear regression and the IC50 values.

FIG. 6 shows the localization of DOX-derived fluorescence in U87 glioblastoma cells after treatment. The cells treated with 20 μM DOX showed fluorescence in the nucleus after only 2 h of treatment, as seen in the upper panel of the image. The fluorescence appeared in red channel. The cells treated with CON1 and CON2 (2, 5, 10 μM) showed perinuclear fluorescence, which means the fluorescence originated from around the nucleus. The lower panels of the image show the fluorescence observed after 72 h of treatment. The nuclei were contrasted with Hoechst 33342 and appeared in blue channel. FIG. 7 depicts the physico-chemical characteristics of CON1 and CON2. The Zetasizer was used to discover the nanoparticle nature of CON1 and CON2, as shown in the upper panel. The size and dispersity of CON1 and CON2 were measured independently three times, and the results are presented in the upper panel. Additionally, the lower panel shows a chemical model of the nanoparticles. Chemical modeling predicted a spontaneous, “protein-like” folding of CON1 and CON2, as a result of strong intramolecular forces.

FIG. 8 shows the TEM characterization of CON1 and CON2.

FIG. 8A illustrates the size and shape of nanoparticles using TEM with a digital camera SIS MegaView Ill and iTEM software.

FIG. 8B shows representative images of the cellular import and subcellular distribution of CON1 or CON2 in U87 cells after 24 h treatment. Both CON1 and CON2 were found to enter into the U87 cells. Depending on the cell section's depth, both large and small nanoparticles were found in the interior of the U87 cells. These nanoparticles were detected on the cell membrane, in the cytoplasm, mitochondria, and predominantly at the level of the nuclear envelope and in the nucleus. Notably, CON1 was more present in the nucleolus. The nucleus is abbreviated as “Nu” and the nucleolus as “No”. The scale bar is 500 nm.

FIG. 9 shows the toxicity profiles of DOX and CON1 in Balb/c mice.

FIGS. 9A, 9B, and 9C illustrate biochemical analysis performed by the Critical Care Panel (900-330).

FIG. 9D shows a part of the whole blood analysis using the scil Vet ABC™Hematology Analyzer. The Balb/c mice were treated with vehicle (10% DMSO and 5% Tween80) in the control group, 7 mg/kg DOX of body weight, and 14 mg/kg CON1 of body weight via intraperitoneal injection (i.p.). Three time points were assessed: 24 h, 48 h, and 72 h. Abbreviations: albumine (ALB), total protein (TP), glucose (GLU), alkaline phosphatase (ALP), alanine aminotransferase (ALT), creatine phosphokinase (CPK), lactate (LAC), blood urea nitrogen (BUN), creatinine (CREA), total bicarbonate (tCO2). FIG. 10 The structure of hybrid compounds of sclareol and doxorubicin (conjugates) CON1 and CON2.

### DETAILED DESCRIPTION OF THE INVENTION

The present invention describes the synthesis of two hybrid compounds (conjugates), CON1 and CON2, containing distinct sclareol derivative ligands, LIG1 and LIG2.

The key synthetic step according to the invention is the oxidative Heck cross-coupling reaction catalyzed by a palladium complex to introduce a 4-formylphenyl group at the C15 position of Sclareol. The resulting product, compound 1 (Scheme 1), is novel. Subsequently, this product undergoes further derivatization. Furthermore, the invention encompasses the synthesis of compounds LIG1 and LIG2 through reductive amination (Scheme 3), which are then transformed into the final products CON1 and CON2 (Scheme 4).

According to the invention, compound 1, ligands LIG1, LIG2, and the hybrid conjugates CON1 and CON2 were synthesized for the first time and characterized. Additionally, the biological activity of compounds LIG1, LIG2, CON1, and CON2 is described.

The synthesis process includes the following reaction steps:
a) Natural product sclareol (SC) reacts with 4-formylphenylboronic acid in the presence of a palladium-based catalyst to yield the corresponding derivative 1 of sclareol in a yield of 80% (Scheme 1). The configuration at the chiral carbon atoms of the sclareol subunit (C-5, C-8, C-10, and C-13) remains unchanged under the applied reaction conditions.

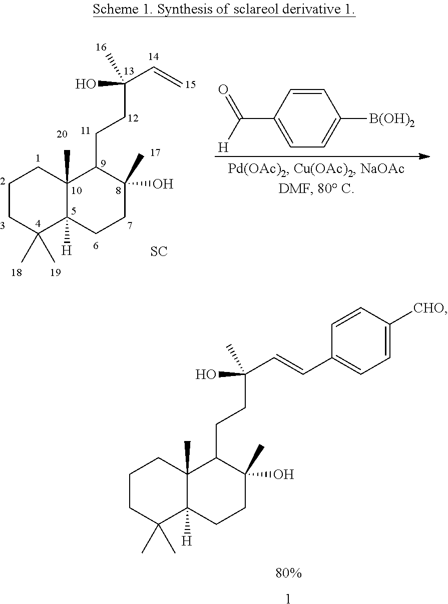

b) Using alkyl diamines, 1,3-diaminopropane, and 1,6-diaminohexane, and succinic anhydride, compounds 2 and 3 were synthesized in good yields (Scheme 2).

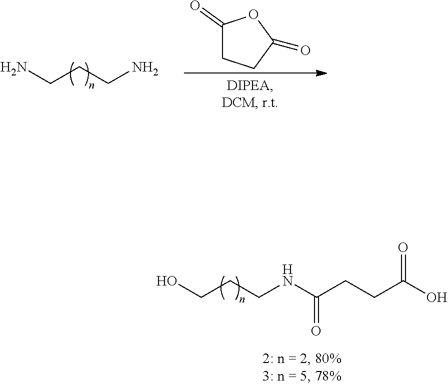

c) Sclareol derivative 1 and compounds 2 and 3 were converted into the corresponding ligands LIG1 and LIG2 in good yields using sodium borohydride in the presence of titanium (IV) isopropoxide via a reductive amination reaction (Scheme 3).

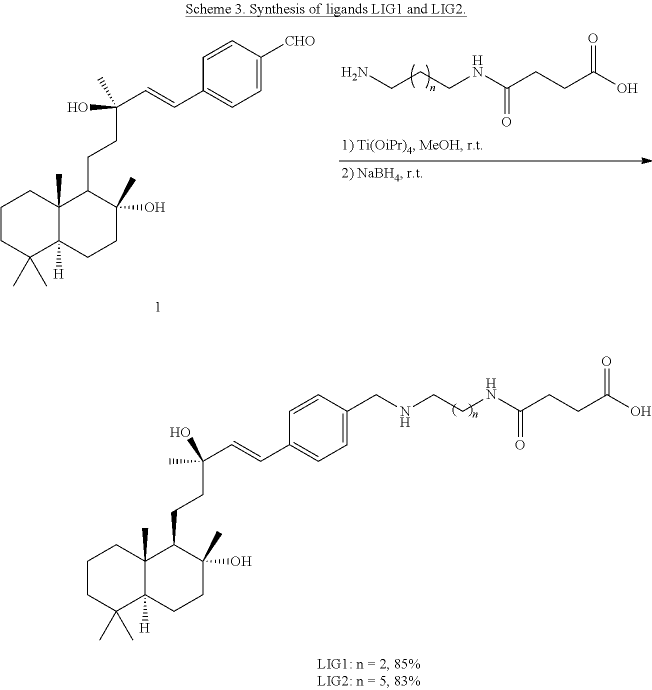

d) The conjugates CON1 and CON2 were obtained by forming an amide bond between doxorubicin and ligands LIG1 and LIG2. To activate the carboxylic functional group of ligands LIG1 and LIG2, 1-hydroxybenzotriazole and 1-ethyl-3-(3-dimethylaminopropyl) carbodiimide were used (Scheme 4).

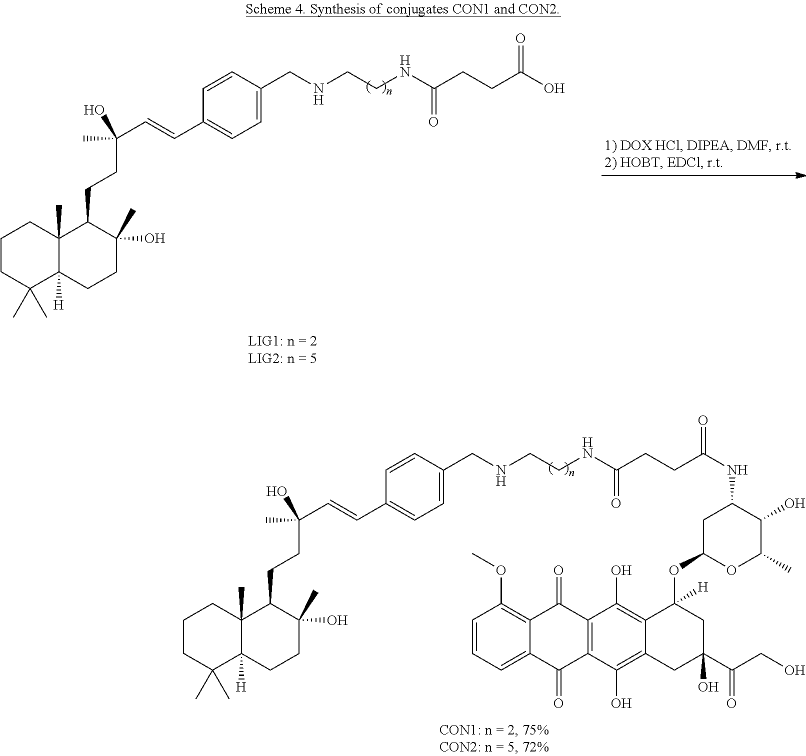

### Examples

The following examples illustrate the synthesis of some compounds of the invention. These examples are not limiting and are provided for illustrative purposes.

Melting points were determined using a Boetius PMHK apparatus and were not corrected. IR spectra were recorded on a Thermo-Scientific Nicolet 6700 FT-IR “diamond crystal” spectrophotometer. Absorption band positions are expressed in cm⁻¹. ¹H NMR and ¹³C NMR spectra were recorded on a Varian spectrometer (at 400 and 100 MHz) in the specified solvent. Chemical shifts are expressed in ppm, coupling constants (J) in Hz, and signal multiplicity is indicated as s (singlet), d (doublet), t (triplet), q (quartet), sext (sextet), dd (doublet of doublets), dq (doublet of quartets), and m (multiplet). HRMS-HESI spectra were recorded on an LTQ Orbitrap XL (Thermo Fisher Scientific Inc., USA) mass spectrometer. Samples were dissolved in pure HPLC-grade CH3CN and directly injected into the instrument. Ionization was performed in positive mode on a heated electrospray ionization probe. The following HESI parameters were used: spray voltage 4.7 kV, evaporation temperature 60° C., drying gas and auxiliary gas flow rates of 24 and 10 (arbitrary units), respectively, capillary voltage 49 V, capillary temperature 275° C., tube lens voltage 80 V, resolution (for m/z 400): 30,000. For thin-layer chromatography, SiO2 and RP-18 plates (Merck) were used. SiO2 (0.018-0.032 mm) was used for “dry-flash” chromatography.

Complete structural characterization of the synthesized compounds was achieved by determining melting points and employing infrared spectroscopy (IC ATR), nuclear magnetic resonance (1D NMR (1H and ¹³C) methods and 2D NMR (COSY (1H-1H Correlation Spectroscopy), NOESY (Nuclear Overhauser Effect Spectroscopy), HSQC (1H-¹³C Heteronuclear Single Quantum Coherence), HMBC (1H-¹³C Heteronuclear Multiple Bond Correlation)) methods), as well as high-resolution mass spectrometry (HRMS).

The purity (HPLC) of the compounds was determined using an Agilent 1200 HPLC system equipped with a Quat Pump (G1311B), Injector (G1329B, 1260 ALS, TCC 1260 (G1316A), and Detector 1260 DAD VL+ (G1315C)). HPLC analyses were performed in two different systems.

Method A: InfinityLab Poroshell 120 CS-C18 4.6×100 0 mm 2.7u, S.N. was used as the stationary phase. The eluent consisted of the following solvents: 0.1% HCOOH in water (A) and MeOH (B). Analyses were performed at the UV maximum of the compound (254 nm) to achieve maximum selectivity.

Compounds were dissolved in MeOH at final concentrations of approximately 1 mg/ml, and the injection volume was 5 μL. The flow rate was 0.6 mL/min. Compounds LIG1, LIG2, CON1, and CON2 were eluted using the following gradient: 0-1 min 95% A, 1-6 min 95% A→5% A, 6-11 min 5% A, 11-14 min 5% A→95% A, 14-15 min 95% A.

Method B: InfinityLab Poroshell 120 CS-C18, 4.6×10⁰ mm, 2.7 μm, with Serial Number (S.N.), was employed as the stationary phase. The eluent consisted of the following solvents: 0.1% HCOOH in water (A) and acetonitrile (ACN) (B). Analyses were conducted at the UV maximum of the compound (254 nm) to achieve maximum selectivity. Compounds were dissolved in methanol (MeOH) at final concentrations of approximately 1 mg/mL, and the injection volume was set at 5 μL. The flow rate was maintained at 0.6 mL/min. Compounds LIG1, LIG2, CON1, and CON2 were eluted using the following gradient: 0-1 min, 95% A; 1-6 min, 95% A→5% A; 6-11 min, 5% A; 11-14 min, 5% A→95% A; 14-15 min, 95% A.

The applied experimental conditions did not impact the stereoisomerism of chiral carbon atoms within the sclareol subunit and doxorubicin.

Example of the synthesis of derivative 1 of sklareol: 4-{(1E,3R)-3-hydroxy-5-[(2R,4aS,8aS)-2-hydroxy-2,5,5,8a-tetramethyldecahydronaphthalen-1-yl]-3-methylpent-1-en-1-yl}benzaldehyde.

In an oxygen-free environment, dimethylformamide (DMF) (1.5 mL), sclareol SC (30 mg, 0.097 mmol, 1 equiv), 4-formylphenylboronic acid (22 mg, 0.146 mmol, 1.5 equiv), Pd(OAc) 2 (2.2 mg, 0.010 mmol, 0.1 equiv), Cu(OAc) 2 (35.3 mg, 0.194 mmol, 2.0 equiv), and NaOAc (23.9 mg, 0.292 mmol, 3 equiv) were added to a round-bottom flask. The reaction mixture was stirred at 80° C. for 2 hours. The reaction mixture was then filtered through a sintered funnel, washed with 3×20 ml of ethyl acetate (EtOAc), and dried over anhydrous MgSO₄. Compound 1 was obtained by purifying the crude product through “dry-flash” chromatography (SiO₂: Hex/EtOAc=7/3) as a yellow oil (32 mg, 80%). ¹H NMR (400 MHz, CDCl₃): δ=9.97 (s, 1H, —CHO), 7.81 (d, J=7.9 Hz, 2H, 2×Ar—H), 7.50 (d, J=7.9 Hz, 2H, 2×Ar—H), 6.69 (d, J=16.0 Hz, 1H, ═CH), 6.46 (d, J=16.2 Hz, 1H, ═CH), 2.45 (brs, 2H, 2×OH), 1.84 (d, J=12.1 Hz, 1H), 1.80-1.72 (m, 2H), 1.68-1.53 (m, 4H), 1.47-1.39 (m, 3H), 1.38 (s, 3H), 1.37-1.33 (m, 1H), 1.29-1.25 (m, 1H), 1.24-1.21 (m, 1H), 1.17 (s, 3H, Me), 1.15-1.07 (m, 1H), 1.02-0.88 (m, 2H), 0.85 (s, 3H, Me), 0.78 (s, 6H, 2×Me) ppm. ¹³C NMR (100 MHz, CDCl₃): δ=191.9, 143.7, 142.0, 135.3, 130.3, 127.0, 125.7, 75.2, 73.7, 61.4, 56.2, 45.1, 44.6, 42.1, 39.9, 39.4, 33.5, 33.4, 27.6, 24.5, 21.6, 20.6, 19.2, 18.5, 15.4 ppm. HRMS (HESI/Orbitrap) m/z: [M+Na]⁺Calcd for C₂₇H₄₁O₃Na⁺435.28697; Found 435.28704. IR (ATR): v=3379, 2927, 2868, 2735, 1698, 1601, 1568, 1461, 1388, 1306, 1266, 1214, 1167, 1133, 1099, 1085, 1065, 1039, 997, 972, 938, 908, 863, 812, 786, 737, 702, 661 cm⁻¹. [α]²⁵, +36.2 (c=0.21 g/mol, MeOH).

### Example of the Synthesis of Compound 2:4-[(6-Aminopropyl)Amino]-4-Oxobutanoic Acid

A solution of succinic anhydride (271 mg, 2.70 mmol) in tetrahydrofuran (THF) (5 mL) was slowly added over 1 hour to a solution of 1,3-diaminopropane (200 mg, 2.70 mmol) in THF (10 mL). The reaction mixture was stirred at room temperature for 2 hours. The reaction was quenched, and the solvent was removed under reduced pressure to yield the crude product. Compound 2 was obtained by purifying the crude product through “dry-flash” chromatography (SiO₂: DCM→DCM/MeOH 1/1→MeOH) as a colorless solid (376 mg, 80%). ¹H NMR (400 MHz, D₂O) δ=3.15 (t, J=6.6 Hz, 2H, —CH₂NHCO-), 2.91-2.82 (m, 2H, —CH₂NH₂), 2.32 (s, 4H, —CH₂COOH and —CH₂CONH), 1.78-1.67 (m, 2H, —CH₂CH₂NH₂). ¹³C NMR (101 MHz, D₂O) δ=180.82, 176.34, 36.84, 35.78, 32.82, 32.15, 26.61.

### Example of the Synthesis of Compound 3:4-[(6-Aminohexyl)Amino]-4-Oxobutanoic Acid

In a solution of succinic anhydride (200 mg, 2.00 mmol) in tetrahydrofuran (THF) (5 mL), a solution of 1,3-diaminohexane (232 mg, 2.00 mmol) in THF (10 mL) was slowly added over 1 hour. The reaction mixture was stirred at room temperature for 2 hours. The reaction was terminated, and the solvent was removed under reduced pressure to obtain the crude product. Compound 3 was obtained by purifying the crude product through “dry-flash” chromatography (SiO₂: DCM→DCM/MeOH 1/1→MeOH) as a colorless solid substance (290 mg, 78%). ¹H NMR (400 MHz, D₂O) δ=3.19 (t, J=6.7 Hz, 2H, —CH₂NHCO—), 3.01 (d, J=7.5 Hz, 2H, —CH₂NH₂), 2.46 (s, 4H, —CH₂COOH and —CH₂CONH), 1.73-1.62 (m, 2H, —CH₂CH₂NH₂), 1.57-1.47 (m, 2H, —CH₂CH₂NHCO—), 1.46-1.36 (m, 4H, —CH₂CH₂CH₂NH₂ and —CH₂CH₂CH₂CH₂NH₂). ¹³C NMR (101 MHz, D₂O) δ=180.80, 175.64, 39.29, 38.95, 33.05, 32.38, 27.89, 26.47, 25.21, 24.99.

### Synthesis of Ligand LIG1: 4-((3-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)propyl)amino)-4-oxobutanoic acid

In a flame-dried round-bottom flask, dry MeOH (4 mL) was added, followed by 4-{(1E,3R)-3-hydroxy-5-[(2R,4aS,8aS)-2-hydroxy-2,5,5,8a-tetramethyldecahydronaphthalen-1-yl]-3-methylpent-1-en-1-yl}benzaldehyde 1 (80 mg, 0.194 mmol) and Ti(Oi-Pr) 4 (235 μL, 0.776 mmol). Then, a solution of amine 2 (101 mg, 0.586 mmol) in dry MeOH (0.5 mL) was slowly added. The reaction mixture was left stirring at room temperature overnight, after which NaBH₄ (13 mg, 0.388 mmol) was added to the solution, and the reaction mixture was further stirred for 2 hours at room temperature. The reaction was quenched by adding water (3 mL), and the reaction mixture was filtered through a silica gel column, followed by thorough washing with DCM. Removal of the solvent under reduced pressure yielded the crude product. Compound LIG1 was obtained after dry-flash chromatography on a silica gel column (SiO₂: DCM→DCM/MeOH (NH₃)=8/2) as a colorless solid (94 mg, 85%) with a melting point of 108-110° C. ¹H NMR (400 MHz, CD₃OD): δ=7.43 (d, J=8.2 Hz, 2H, 2×Ar—H), 7.37 (d, J=8.3 Hz, 2H, 2×Ar-H), 6.60 (d, J=16.1 Hz, 1H, ═CH), 6.36 (d, J=16.2 Hz, 1H, ═CH), 3.88 (s, 2H, Ar-CH₂-N), 3.26 (t, J=6.5 Hz, 2H, —CH₂NHCO—), 2.80 (t, J=7.2 Hz, 2H, —CH₂NHCH₂Ar—), 2.46-2.35 (m, 4H, —CH₂COOH iand —CH₂CONH), 1.88-1.79 (m, 2H), 1.78-1.73 (m, 2H), 1.72-1.60 (m, 4H), 1.60-1.50 (m, 1H), 1.49-1.37 (m, 4H), 1.36 (s, 3H, Me), 1.36-1.28 (m, 1H), 1.25-1.15 (m, 1H), 1.15-1.12 (m, 1H), 1.12 (s, 3H, Me), 1.03-0.93 (m, 2H), 0.89 (s, 3H, Me), 0.83 (s, 3H, Me), 0.82 (s, 3H, Me) ppm. ¹³C NMR (101 MHz, CD₃OD): δ=180.67, 176.74, 164.67, 162.52, 138.83, 130.37, 127.65, 127.57, 75.17, 74.35, 62.73, 57.56, 53.45, 49.85, 49.28, 47.08, 46.56, 45.17, 43.25, 41.18, 40.56, 37.44, 34.64, 34.18, 33.90, 33.88, 29.19, 27.82, 23.97, 21.95, 21.54, 20.84, 19.50, 16.05. HRMS (HESI/Orbitrap) m/z: [M+H]⁺Calcd for C₃₄H₅₅O₅N₂ 571.41055; Found 571.41120. IR (ATR): v=3292, 2929, 2867, 1640, 1562, 1459, 1388, 1301, 1270, 1219, 1188, 1157, 1132, 1085, 1033, 996, 970, 938, 908, 865, 640 cm⁻¹. HPLC purity, method E: t_{R}=7.608 min, area 99.42%. Method B: t_{R}=6.137 min, area 99.07% (λ=254 nm). [α]_{D}²⁵+0.054 (c=3.3×10³ g/mol, MeOH).

### Synthesis of Ligand LIG2: 4-((6-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)hexyl)amino)-4-oxobutanoic acid

In a flame-dried round-bottom flask, dry MeOH (4 mL) was added, followed by 4-{(1E,3R)-3-hydroxy-5-[(2R,4aS,8aS)-2-hydroxy-2,5,5,8a-tetramethyldecahydronaphthalen-1-yl]-3-methylpent-1-en-1-yl}benzaldehyde 1 (95 mg, 0.231 mmol) and Ti(Oi-Pr) 4 (262 μL, 0.924 mmol). Then, a solution of amine 3 (150 mg, 0.694 mmol) in dry MeOH (0.5 mL) was slowly added. The reaction mixture was left stirring at room temperature overnight, after which NaBH₄ (17.4 mg, 0.462 mmol) was added to the solution, and the reaction mixture was further stirred for 2 hours at room temperature. The reaction was quenched by adding water (3 mL), and the reaction mixture was filtered through a silica gel column, followed by thorough washing with DCM. Removal of the solvent under reduced pressure yielded the crude product. Compound LIG2 was obtained after dry-flash chromatography (SiO₂: DCM→DCM/MeOH (NH₃)=8/2) as a colorless solid (141 mg, 83%) with a melting point of 124-126° C. ¹H NMR (400 MHz, CD₃OD): δ=7.49 (d, J=8.1 Hz, 2H, 2×Ar—H), 7.44 (d, J=8.2 Hz, 2H, 2×Ar—H), 6.61 (d, J=16.1 Hz, 1H, ═CH), 6.41 (d, J=16.1 Hz, 1H, ═CH), 4.14 (s, 2H, Ar-CH₂-N), 3.19 (t, J=6.3 Hz, 2H, —CH₂NHCO—), 2.99 (t, J=7.7 Hz, 2H, —CH₂NHCH₂Ar—), 2.50-2.35 (m, 4H, —CH₂COOH and —CH₂CONH), 1.93-1.77 (m, 2H), 1.75-1.56 (m, 6H), 1.55-1.46 (m, 3H), 1.44-1.37 (m, 6H), 1.35 (s, 3H, Me), 1.35-1.28 (m, 1H), 1.22-1.16 (m, 1H), 1.15-1.10 (m, 1H), 1.11 (s, 3H, Me), 1.00-0.91 (m, 2H), 0.88 (s, 3H, Me), 0.82 (s, 3H, Me), 0.81 (s, 3H, Me) ppm. ¹³C NMR (101 MHz, CD₃OD): δ=180.53, 176.00, 140.04, 139.84, 132.01, 131.16, 127.95, 127.20, 75.17, 74.35, 62.73, 57.56, 54.80, 52.08, 49.29, 48.18, 47.03, 45.17, 43.25, 41.17, 40.55, 39.75, 34.61, 34.18, 33.94, 33.90, 29.84, 27.92, 27.06, 26.83, 26.69, 23.95, 21.94, 21.53, 20.82, 19.51, 16.05. HRMS (HESI/Orbitrap) m/z: [M+H]⁺Calcd for C₃₇H₆₁O₅N₂613.45750; Found 613.45804. IR (ATR): v=3289, 3089, 2927, 2862, 1635, 1561, 1437, 1387, 1300, 1268, 1216, 1178, 1132, 1084, 1032, 995, 970, 938, 908, 864, 802, 724, 642, 563 cm⁻¹. HPLC purity, method A: t_{R}=7.683 min, area 99.32%. Method B: t_{R}=6.191 min, area 99.11% (λ=254 nm). [α]_{D}²⁵+0.054 (c=1.3×10-3 g/mol, MeOH).

### Synthesis of CON1: N1-((25,35,45,6R)-3-hydroxy-2-methyl-6-(((15,3S)-3,5,12-trihydroxy-3-(2-hydroxyacetyl)-10-methoxy-6,11-dioxo-1,2,3,4,6,11-hexahydrotetracen-1-yl)oxy)tetrahydro-2H-pyran-4-yl)-N4-(3-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)propyl) succinamide

In a solution of doxorubicin hydrochloride (21 mg, 0.036 mmol) in dry DMF (500 ML), diisopropylethylamine (6 μL, 0.050 mmol) was added. The addition of DIEA caused the solution to change color from light red to dark red. The reaction mixture was left stirring at room temperature for 30 minutes under argon, followed by the addition of compound LIG1 (21 mg, 0.036 mmol). After cooling the reaction mixture in an ice bath (0° C.), solutions of HOBT (6.1 mg, 0.050 mmol) and EDCl (9.4 mg, 0.050 mmol) in dry DMF (100 μL each) were added sequentially. The reaction mixture was stirred for 30 minutes at 0° C. and then for 18 hours at room temperature in the dark. The reaction progress was monitored using an Agilent 1200 HPLC system equipped with a reverse-phase analytical InfinityLab Poroshell 120 CS-C18 column (4.6×10⁰ mm, 2.7u, S.N. USKBM01053). The mobile phase consisted of a mixture of 0.1% aqueous formic acid and ACN with a programmed isocratic and gradient elution: 0-1 min, 5% ACN; 1-6 min, 5%→95% ACN; 6-12 min, 95% ACN; 12-14 min, 95% A→5% ACN; 14-15 min, 5% ACN. The flow rate was 0.6 mL/min. Signal detection was performed in the wavelength range of 254/280 nm using a DAD detector. After 12 hours, the reaction was stopped, and the reaction mixture was concentrated under reduced pressure. The crude reaction mixture was purified on an HPLC-DAD system using a semi-preparative ZORBAX Eclipse XDB-C18 column (9.4× 250 mm, 5u, S.N. USSY004729) (DAD detector: 254 nm). The mobile phase consisted of a mixture of 0.1% aqueous formic acid and MeOH. The flow rate was 3.5 mL/min, and gradient elution was carried out as follows: 0-1 min, 5% MeOH; 1-6 min, 5→100% MeOH; 6-10 min, 100% MeOH, resulting in the isolation of CON1 (Rf=8.31 min). The solvent was removed under reduced pressure, and the residue was dissolved in DCM. The organic layer was washed several times with aqueous NaHCO₃ solution, saturated aqueous NaCl solution, and dried over anhydrous Na₂SO₄. After filtration, the solvent was removed by rotary evaporation under reduced pressure. Compound CON1 was obtained as a dark red powder (29.7 mg, 75%) that softens at 178-180° C. ¹H NMR (400 MHz, CDCl₃+CD₃OD): δ=7.90 (d, J=6.7 Hz, 1H, H-C₄Ar(dox)), 7.70 (t, J=8.1 Hz, 1H, H-C₃Ar(dox)), 7.32 (d, J=8.4 Hz, 1H, H-C₂Ar(dox)), 7.25-7.10 (m, 4H, 4×H-Ar (scl)), 6.40 (d, J=16.4 Hz, 1H, ═CH), 6.19 (d, J=16.3 Hz, 1H, ═CH), 5.40-5.35 (šs, 1H, H-2′), 5.17-5.10 (m, 1H, H-C₁₀Ar(dox)), 4.61 (s, 2H, CH₂—OH), 4.02 (d, 1H, J=6.7 Hz, H-6′), 4.00-3.90 (m, 1H, H-4′), 3.97 (s, 3H, -O-CH₃), 3.86 (s, 2H, Ar-CH₂—NH), 3.50-3.45 (brs, 1H, H-5′), 3.20-3.00 (m, 3H, CH₂NHCO—, H-C₇Ar(dox), 3.00-2.85 (m, 1H, H-C₇Ar(dox), 2.80-2.70 (m, 2H, —CH₂NHCH₂Ar—), 2.40-2.10 (m, 5H, -NHCOCH₂CH₂CONH and H-C₇Ar(dox)), 2.05-1.90 (m, 1H, H-C₇Ar(dox)), 1.85-1.65 (m, 4H, H-C₃Ar(dox)), 1.65-1.35 (m, 6H, H-C₃Ar(dox)), 1.30-1.20 (m, 3H), 1.23 (s, 3H, Me), 1.05-0.90 (m, 2H), 1.16 (d, J=6.7 Hz, 3H, 6′—CH₃), 1.04 (s, 3H, Me), 0.90-0.75 (m, 2H), 0.74 (s, 3H, Me), 0.68 (s, 3H, Me), 0.67 (s, 3H, Me). ¹³C NMR (101 MHz, CDCl₃+CD₃OD): δ=213.65 (C₁₃═O), 187.14 (C₁₂═O), 186.71 (C₅═O), 174.33 (NHC═O), 172.20 (NHC═O), 161.00 (C1), 155.92 (C6), 155.19 (C11), 139.42 (═CH), 138.48, 135.88 (C3), 135.36 (C4a), 133.86 (10a), 133.60 (C6a), 129.82 (2×Ar (scl)), 126.83 (2×Ar (scl)), 126.53, 125.26 (═CH), 120.71 (C12a), 119.75 (C4), 118.61 (C2), 111.46 (5a), 111.25 (11a), 100.69 (C2′), 77.36, 76.33 (C8), 74.49, 73.06, 69.37 (C10), 68.28 (C5′), 67.41 (C6′), 65.05 (C14), 61.38, 56.54 (O-CH₃), 56.05, 51.31 (Ar-CH₂—NH), 45.55, 45.00, 44.50 (NH—C4′), 43.68, 41.90, 39.66, 39.18, 36.03, 35.69 (C9), 33.54 (C7), 33.21 (Me), 33.11, 31.03, 29.60, 29.28 (C3′), 26.20, 23.64 (Me), 21.32 (Me), 20.32 (Me), 18.91, 18.34, 16.64 (6′—CH₃), 15.30 (Me). HRMS (HESI/Orbitrap) m/z: [M+H]⁺Calcd for C61H₈₂O₁₅N₃1096.57405; Found 1096.57532. IR (ATR): v=3360, 2926, 1724, 1618, 1577, 1444, 1411, 1387, 1284, 1236, 1209, 1170, 1116, 1083, 1018, 985, 794, 765, 611, 463 cm⁻¹. HPLC purity, method A: t_{R}=8.309 min, area 96.13%. Method B: t_{R}=6.688 min, area 95.32% (λ=254 nm). [α]_{D}²⁵+0.012 (c=1.7×10⁻⁴ g/mol, MeOH).

Synthesis of CON2: N1-((25,35,4S,6R)-3-hydroxy-2-methyl-6-(((1S,3S)-3,5,12-trihydroxy-3-(2-hydroxyacetyl)-10-methoxy-6,11-dioxo-1,2,3,4,6,11-hexahydrotetracen-1-yl)oxy)tetrahydro-2H-pyran-4-yl)-N4-(6-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)hexyl) succinamideln a solution of doxorubicin hydrochloride (15 mg, 0.052 mmol) in dry DMF (500 μL), diisopropylethylamine (13 μL, 0.036 mmol) was added. The addition of DIEA caused the solution to change color from light red to dark red. The reaction mixture was left stirring at room temperature for 30 minutes under argon, followed by the addition of compound LIG2 (16 mg, 0.026 mmol). After cooling the reaction mixture in an ice bath (0° C.), solutions of HOBT (8.9 mg, 0.036 mmol) and EDCl (14 mg, 0.036 mmol) in dry DMF (100 μL each) were added sequentially. The reaction mixture was stirred for 30 minutes at 0° C. and then for 18 hours at room temperature in the dark. The reaction progress was monitored using an Agilent 1200 HPLC system equipped with a reverse-phase analytical InfinityLab Poroshell 120 CS-C18 column (4.6×10⁰ mm, 2.7u, S.N. USKBM01053). The mobile phase consisted of a mixture of 0.1% aqueous formic acid and ACN with a programmed isocratic and gradient elution: 0-1 min, 5% ACN; 1-6 min, 5%→95% ACN; 6-12 min, 95% ACN; 12-14 min, 95% A→5% ACN; 14-15 min, 5% ACN. The flow rate was 0.6 mL/min. Signal detection was performed in the wavelength range of 254/280 nm using a DAD detector. After 12 hours, the reaction was stopped, and the reaction mixture was concentrated under reduced pressure. The crude reaction mixture was purified on an HPLC-DAD system using a semi-preparative ZORBAX Eclipse XDB-C18 column (9.4×250 mm, 5u, S.N. USSY004729) (DAD detector: 254 nm). The mobile phase consisted of a mixture of 0.1% aqueous formic acid and MeOH. The flow rate was 3.5 mL/min, and gradient elution was carried out as follows: 0-1 min, 5% MeOH; 1-6 min, 5→100% MeOH; 6-10 min, 100% MeOH, resulting in the isolation of CON2 (Rf=8.33 min). The solvent was removed under reduced pressure, and the residue was dissolved in DCM. The organic layer was washed several times with aqueous NaHCO₃ solution, saturated aqueous NaCl solution, and dried over anhydrous Na₂SO₄. After filtration, the solvent was removed by rotary evaporation under reduced pressure. Compound CON2 was obtained as a dark red powder (21.3 mg, 75%) that softens at 170-172° C. ¹H NMR (400 MHz, CDCl₃+CD₃OD): δ=7.86 (d, J=8.7 Hz, 1H, H-C₄Ar(dox)), 7.65 (t, J=8.1 Hz, 1H, H-C₃Ar(dox)), 7.28 (d, J=8.7 Hz, 1H, H-C₂Ar(dox)), 7.21 (d, J=8.2 Hz, 2H, 2×H-Ar (scl)), 7.14 (d, J=7.9 Hz, 2H, 2×H-Ar (scl)), 6.38 (d, J=16.4 Hz, 1H, ═CH), 6.14 (d, J=16.1 Hz, 1H, ═CH), 5.35-5.30 (brs, 1H, H-2′), 5.13-5.07 (m, 1H, H-C₁₀Ar(dox)), 4.59 (s, 2H, CH₂-OH), 3.99-3.95 (m, 1H, H-6′), 3.95-3.85 (m, 1H, H-4′), 3.91 (s, 3H, -O-CH₃), 3.74 (s, 2H, Ar-CH₂—NH), 3.40-3.35 (brs, 1H, H-5′), 3.12-3.02 (m, 1H, H-C₇Ar(dox)), 2.96 (t, J=6.6 Hz, 2H, —CH₂NHCO—), 2.92-2.82 (m, 1H, H-C₇Ar(dox), 2.57 (t, J=7.3 Hz, 2H, —CH₂NHCH₂Ar—), 2.40-2.10 (m, 5H, -NHCOCH₂CH₂CONH and H-CgAr(dox)), 2.10-1.90 (m, 1H, H-C₇Ar(dox)), 1.85-1.75 (m, 1H, H-C3: Ar(dox)), 1.70-1.50 (m, 4H, H-C₃Ar(dox)), 1.50-1.35 (m, 6H), 1.35-1.15 (m, 6H), 1.18 (s, 3H, Me), 1.15-1.05 (m, 5H), 1.05-0.95 (m, 2H), 1.12 (d, J=6.6 Hz, 3H, 6′—CHs), 0.85-0.70 (m, 2H), 0.98 (s, 3H, Me), 0.85-0.70 (m, 2H), 0.69 (s, 3H, Me), 0.63 (s, 3H, Me), 0.61 (s, 3H, Me). ¹³C NMR (101 MHz, CDCl₃+CD₃OD): δ=213.56 (C₁₃═O), 187.14 (C₁₂═O), 186.67 (C₅═O), 172.89 (NHC═O), 172.30 (NHC═O), 160.94 (C₁), 155.88 (C₁₁), 155.17 (C₆), 138.95 (═CH), 135.83 (C₃), 135.30 (C₄a), 133.92 (C₁₀a), 133.62 (C₆a), 129.39 (2×Ar (scl)), 126.67 (2×Ar (scl)), 125.33 (═CH), 120.63 (C12a), 119.65 (C4), 118.58 (C2). 111.41 (C11a), 111.17 (C5a), 100.64 (C2′), 77.36, 76.24 (C8), 74.32, 72.96, 69.33 (C10), 68.22 (C5′), 67.29 (C6′), 64.94 (C14), 61.21, 56.40 (O-CH₃), 55.98, 51.48 (Ar-CH₂—NH), 45.47 (NH—C4′), 44.98, 43.55, 41.81, 39.58, 39.10, 38.93, 35.70 (C9), 33.39 (C7), 33.07 (Me), 33.00, 31.18, 31.02, 29.48, 29.16 (C3′), 28.52, 26.07 (Me), 25.94, 23.46 (Me), 21.18 (Me), 20.21, 18.92, 18.23, 16.50 (6′—CH₃), 15.18 (Me). HRMS (HESI/Orbitrap) m/z: [M+H]⁺Calcd for C₆₄H₈₈O₁₅N₃1138.62100; Found 1138.62158. IR (ATR): v=3314, 2932, 2865, 1721, 1582, 1444, 1412, 1385, 1347, 1285, 1209, 1115, 1084, 1017, 986, 792, 764, 587, 478 cm⁻¹. HPLC purity, method A: t_{R}=8.336 min, area 98.18%. Method B: t_{R}=6.735 min, area 98.75% (λ=254 nm). [α]²⁵, +0.019 (c=1.7×10-4 g/mol, MeOH).

### Biological Tests

### Materials and Methods

### Compounds

The compounds used in this study are doxorubicin (DOX, Sigma-Aldrich, Germany), sclareol (SC), and hybrid compounds (conjugates) CON1 and CON2, as well as their corresponding ligands (LIG1 and LIG2). All compounds were dissolved as 20 mM stocks in dimethyl sulfoxide (DMSO) and stored as aliquots at −20° C. Prior to treatment, the compounds were dissolved in sterile deionized water.

### Chemicals and Reagents

The following chemicals and reagents were used in the experimental work: rhodamine 123 (Rho123), Hoechst 33342, DMSO, 3-[4,5-dimethylthiazol-2-yl]-2,5-diphenyltetrazolium bromide (MTT) (Sigma-Aldrich Chemie GmbH, Germany), minimal essential medium (MEM), RPMI 1640 medium, Dulbecco's modified minimal essential medium (DMEM), fetal bovine serum (FBS), a mixture of antibiotics penicillin-streptomycin (Capricorn Scientific, Germany), trypsin/EDTA (Biological Industries, USA), L-glutamine, a mixture of antibiotics and antimycotics: penicillin, streptomycin, amphotericin B (Gibco, ThermoFisher Scientific, USA), MEM non-essential amino acids (Biowest, USA).

### Cell Lines

U87 human glioblastoma cell line, DLD1 human colorectal carcinoma cell line, and NCI-H460, human non-small cell lung carcinoma cell line were obtained from the American Type Culture Collection (ATCC, USA). U87-TxR resistant human glioblastoma cell line was obtained by successive exposure of U87 cells to increasing concentrations of paclitaxel (100-300 nM) over six to nine months and is characterized by high expression of P-glycoprotein. DLD1-TxR resistant human colorectal carcinoma cell line was obtained by selection from DLD1 cell line by exposure to gradually increasing concentrations of paclitaxel (60-600 nM) over a period of ten months and is also characterized by higher expression of P-glycoprotein compared to the parental cell line. NCI-H460/R resistant human non-small cell lung carcinoma cell line was obtained by successive exposure of NCI-H460 cells to increasing concentrations of DOX over three months and is characterized by extremely high expression of P-glycoprotein. U251 human glioblastoma cell line and MRC-5 human lung fibroblast cell line were obtained from the European Collection of Authenticated Cell Cultures (ECACC, UK). U87, U87-TxR, and MRC-5 cell lines were grown in MEM medium supplemented with 10% FBS, 1% L-glutamine, 1% antibiotic mixture of penicillin and streptomycin, and 1% non-essential amino acids. NCI-H460, NCI-H460/R, DLD1, and DLD1-TxR were grown in RPMI 1640 medium supplemented with 10% FBS, 1% L-glutamine, and 1% antibiotic-antimycotic mixture. U251 cell line was grown in DMEM medium supplemented with 10% FBS, 1% L-glutamine, and 1% antibiotic mixture of penicillin and streptomycin. All cell lines were grown in an incubator at 37° C., in a humid atmosphere with 5% CO₂.

Cell passage was performed after reaching 80-90% confluence in 25 cm² and 75 cm² flasks

(ThermoFisher Scientific, USA), using 0.25% trypsin/EDTA. After trypsinization, the cells were counted using a Burker-Turk hemocytometer on an inverted microscope. To determine the number of cells, 10 μL of the cell suspension was injected into two grooved chambers of the hemocytometer. Four fields were counted in both chambers. To calculate the total number of cells per ml, the following formula was used: average number of cells per square chamber x cell dilution x 104 (chamber factor). After counting, the cells were sown at an appropriate density for further experimentation or maintenance in culture (at a density of 8000 cells/cm² for NCI-H460, NCI-H460/R, DLD1, and DLD1-TxR, and 16,000 cells/cm² for U87, U87-TxR, U251, and MRC-5).

Cytotoxic Effects of SC, DOX, their Combination, and their Novel Hybrids (CON1 and CON2)

The MTT test was used to evaluate the effect of the compounds on cell viability. The test relies on the reduction of tetrazolium salt (MTT) to formazan, which is catalyzed by the mitochondrial enzyme succinate dehydrogenase. The activity of succinate dehydrogenase, which is indicative of mitochondrial respiration, is taken as an indirect measure of the metabolic activity of the cell, and thus its viability. The MTT-formazan produced is a purple-colored product, whose absorbance is measured at a wavelength of 570 nm.
a) To determine the inhibition of cell growth by SC, different cell lines were exposed to increasing concentrations of SC (5, 10, 25, 50, and 100 μM). The cells were seeded in 96-well microtiter plates at a density of 2,000 cells per well for NCI-H460, NCI-H460/R, DLD1 and DLD1-TxR, and 4,000 cells per well for MRC-5, U87, U87-TxR, and U251 in 100 μL of the appropriate medium. Control cells that received no treatment were also seeded at the same time. After 24 h for adaptation, the cells were treated with various concentrations of SC. After 72 h of treatment, 0.2 mg/ml MTT was added to each well in the appropriate cell culture medium. After 4 h of incubation at 37° C. with 5% CO₂, the medium was removed, and 100 μL of DMSO were added to each well, to dissolve the formazan. The absorbance of the samples was measured at 570 nm using a Multiskan Sky Microplate spectrophotometer (ThermoFisher Scientific, USA). The results were analyzed using GraphPad Prism 8.0.2. software, and the IC50 values were determined by non-linear regression.
b) To test the interaction between SC and DOX in terms of cytotoxicity in different cell lines, cells were seeded in 96-well microtiter plates at a density of 2,000 cells per well for NCI-H460, NCI-H460/R, DLD1 and DLD1-TxR, and 4,000 cells per well for MRC-5, U87, U87-TxR, and U251 in 100 μl of the appropriate medium. SC (10 and 20 μM) was combined with increasing concentrations of DOX (20, 50, 100, 200, and 500 nM). The cells were treated with the simultaneous combinations for 72 h. After this period, 0.2 mg/ml MTT was added to each well, followed by 4 hours of incubation at 37° C. with 5% CO₂. The medium was then removed, and DMSO was added to each well to dissolve the formazan.

The absorbance of the samples was measured at 570 nm using a Multiskan Sky Microplate spectrophotometer.

The analysis was carried out using GraphPad Prism software version 8.0.2 to determine the nature of the interaction, namely synergistic, antagonistic, or additive, between compounds. This was done using the computer software CalcuSyn, which is based on the combination index method. The method takes into account the concentration of compounds, the individual effect of each compound, as well as the effect of two compounds in combination. The values of the combination indices (CI) describe the nature of the interaction: Cl<0.9 indicates synergism, Cl>1.1 indicates antagonism, and Cl=0.9-1.1 indicates an additive effect.
c) To compare the resistance and selectivity profiles among SC, DOX, CON1, and CON2, human glioblastoma cell lines U87, U87-TxR, and MRC-5 were seeded at a density of 4,000 cells per well in 100 μL of the appropriate medium. In contrast, NCI-H460, NCI-H460/R, and MRC-5 were seeded at a density of 2,000 cells per well in 100 μL of the proper medium. The effect of SC derivatives LIG1 and LIG2 was also examined. The range of concentrations differed among the cell lines and the compounds. For LIG1 and LIG2, the range of concentrations for all cell lines was 50, 100, 200, 300, and 400 μM, while the same range was used for the MRC-5 in the case of SC, CON1, and CON2. DOX concentrations ranged 0.1, 0.25, 0.5, 1, and 2.5 μM for U87 and NCI-H460, and 0.25, 0.5, 1, 2.5, and 5 μM for U87-TxR, NCI-H460/R, and MRC-5. Five concentrations used for SC, CON1, and CON2 treatments varied in the range 1, 2.5, 5, 10, 25, 50, and 100 μM. The treatment lasted 72 h. After the treatment period, 0.2 mg/ml MTT in the appropriate cell culture medium was added to each well.

After 4 h of incubation at 37° C. with 5% CO₂, the medium was removed, and DMSO was added to each well to dissolve the formazan. The absorbance of the samples was measured at 570 nm on a Multiskan Sky Microplate spectrophotometer. The results were analyzed using GraphPad Prism 8.0.2. software and the IC50 values were determined by non-linear regression.

### The Examination of P-Glycoprotein Substrate Accumulation (DOX and Rhodamine 123)

a) First, DOX accumulation test was performed on three glioblastoma cell lines, U87, U87-TxR, and U251. Cells were trypsinized and counted, and then 100,000 cells per sample were collected in a tube for flow cytometry and resuspended in 500 μL of culture medium. Cells were treated with 20 μM and 50 M SC. Immediately after SC treatment, cells were treated with 20 μM DOX, and samples were incubated at 37° C. in a humid atmosphere with 5% CO₂ for 60 min. After the accumulation period, the samples were centrifuged, washed twice with cold Phosphate Buffered Saline (PBS), and finally resuspended in 1 mL of PBS. The fluorescence of the samples was read on the FL2 (red channel) of a cytofluorimeter (Partec, Munster, Germany), and the results were analyzed with the software package Summit 4.3.
b) Secondly, DOX and rhodamine 123 accumulation test was performed on non-small cell lung carcinoma cell lines NCI-H460 and NCI-H460/R. Cells were trypsinized and counted, and then 100,000 cells per sample were collected in a tube for flow cytometry and resuspended in 500 μl of culture medium. Cells were treated with 50 μM SC. Immediately after treatment, 5 μM Rho123 was added to one group of samples and the other 20 μM DOX. They were incubated at 37° C. in a humidified atmosphere with 5% CO₂ for 30 min or 60 min, respectively. After the accumulation period, the samples were centrifuged, washed twice with cold PBS, and finally resuspended in 1 ml of PBS. The samples were analyzed using a flow cytometer, reading for rhodamine 123 on FL1 (green channel) and DOX on FL2 (red channel). Summit 4.3 software was used to analyze the results.

### Intracellular Localization of Conjugates (CON1 and CON2) and DOX

The study investigated the localization of fluorescence originating from DOX on a sensitive glioblastoma cell line, U87. The cells were seeded at a density of 24,000 cells per well in a 24-well plate (ThermoFisher Scientific, USA) in 600 μL of the appropriate growth medium. After 24 h, the cells were treated with growing concentrations of CON1 and CON2 (2, 5 and 10 μM) for 72 h. Two wells with untreated cells were used, one as an untreated control and the other treated with 20 μM DOX for 2 h immediately before imaging. The cells were stained with Hoechst 33342 for 15 min. Thereafter, all wells were fixed with 4% paraformaldehyde for 15 min, washed with PBS and imaged on the ZOE Fluorescent Cell Imager (BIO-RAD, USA) in the blue and red channels.

### Physico-Chemical Characteristics of CON1 and CON2

The Malvern Zetasizer Nano ZS (Malvern Instruments, United Kingdom) was used to analyze the physico-chemical stability characteristics of conjugates. The measurement range was from 0.6 nm up to 6 mm. The mean size of conjugates, the polydispersity index (PDI) and zeta potential were measured. All measurements were taken at a temperature of 25° C. and each sample was diluted 100 times with ultrapure water. The measurements were repeated three times, and the results were presented as the mean value. PDI is a parameter used to define the size range of particles. The term “polydispersity” (or “dispersity” as recommended by IUPAC) describes the degree of non-uniformity of a size distribution of particles. Zeta potential represents the surface charge of nanoparticles and indicates their long-term stability.

### Chemical Modeling

Prediction of pKa was performed using Epik v5.7 from Schrödinger Suite 2021-3 [Schrödinger Release 2021-3: Epik, Schrödinger, LLC, New York, NY, 2021]. Conformational search was performed using Conformational Search from MacroModel v13.3 module from Schrödinger Suite 2021-3 [Schrödinger Release 2021-3: MacroModel, Schrödinger, LLC, New York, NY, 2021], with OPLS4 force field, water as a solvent and mixed torsional/low/moode sampling method. Number of steps was 1000 and energy window for saving the structure (cutoff) was 21 kj/mol.

### Transmission Electron Microscopy for CON1 and CON2 Characterisation and Intracellular Localization

a) An analysis of the nanoparticles nature of CON1 and CON2 was conducted using transmission electron microscopy. To prepare for analysis, CON1 or CON2 were diluted 100 times with ultrapure water and Formvar or carbon-coated glow-discharged nickel grids were placed on top of the drops and left for 2-5 min to absorb excess fluid. The particles were examined on a Philips CM12 transmission electron microscope (Philips/FEI, Netherlands) operating at 80 kEV and equipped with the digital camera SIS MegaView III (Olympus Soft Imaging Solutions, Germany). The diameter of CON1 and CON2 particles was measured using iTEM software.
b) For the transmission electron analysis of CON1 and CON2 U87 cell import, 2,000,000 U87 cells were treated 24 h with 5 μM CON1 or CON2, while untreated cells served as controls. Immediately after treatment, cells were rinsed with PBS, fixed with 2.5% glutaraldehyde in 0.1 M cacodylate buffer (pH 7.2) for 2 h at 4° C., and rinsed again in cacodylate buffer. The cells were then postfixed in 2% osmium tetroxide in the same buffer, dehydrated using increasing concentrations of ethanol, and embedded in resin. Semi-fine sections were used for electron microscopy treatment, and ultra-thin sections of cells were obtained using a Leica UC6 ultramicrotome (Leica Microsystems, Germany) and mounted on copper grids. Over 20 cell sections were examined on a Philips CM12 transmission electron microscope equipped with the digital camera SIS MegaView III. The resulting electron micrographs were used for overall U87 cell morphological analysis and CON1 or CON2 cell import analysis.

### Comparative Toxicology Study of DOX and CON1 in Balb/c Mice

Male Balb/c mice, aged 7-11 weeks, were sourced from the EL-42BIO/Br-01 (Laboratory of Pharmacology, School of Medicine, University of Thessaly, Larissa, Greece). The mice were kept in an environment with a 12-hour light/dark cycle, humidity of around 50-60%, and a temperature of 20-22° C. They had free access to food and water. The handling and experimentation of the mice were conducted in compliance with the Greek laws (PD 56/2013 and Circular 2215/117550/2013) and the European Union guidelines (2013/63/EU). The protocol was approved by the IACUC and Greek authorities (License no. 58376/13.02.2023). The mice were divided into three groups with two mice per group, amounting to a total of 18 mice. They were treated as follows: Group 1 received vehicle (10% DMSO and 5% Tween80); Group 2-DOX received 7 mg/kg of body weight via intraperitoneal injection (i.p.); Group 3 received CON1 at 14 mg/kg of body weight (to achieve a DOX concentration of 7 mg/kg of body weight), i.p. The study took place over three time points: 24 h, 48 h and 72 h. At the end of each period, blood samples were collected from the mice's tails and cheeks, and the mice were euthanized by CO₂ inhalation followed by cervical dislocation. Fresh blood samples were analyzed using the scil Vet ABC™Hematology Analyzer (scil animal care company GmbH, Germany), while sera from all samples were extracted and analyzed using the Skyla VB1 Veterinary Clinical Chemistry Analyzer (Skyla Corporation, Taiwan) with the Critical Care Panel (900-330) (Skyla Corporation, Taiwan).

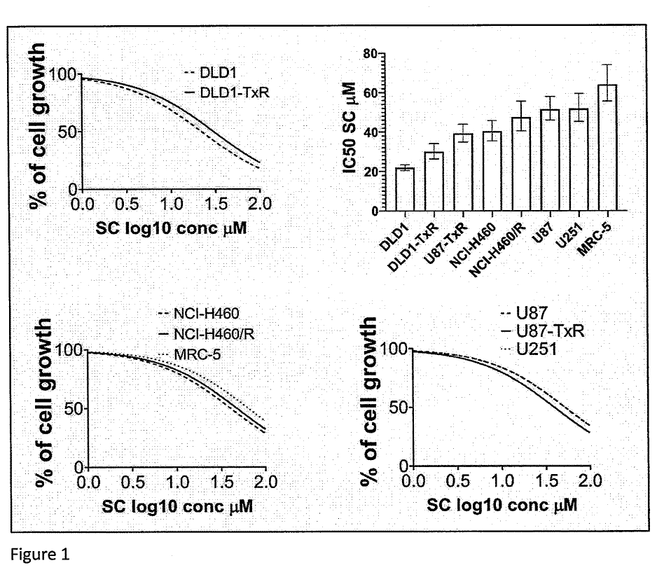

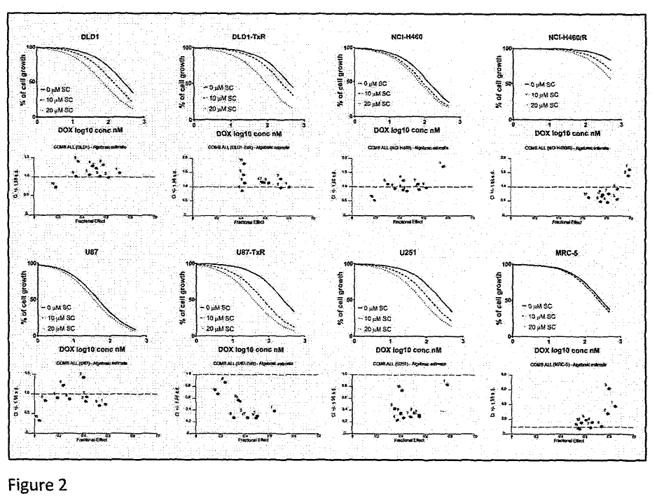

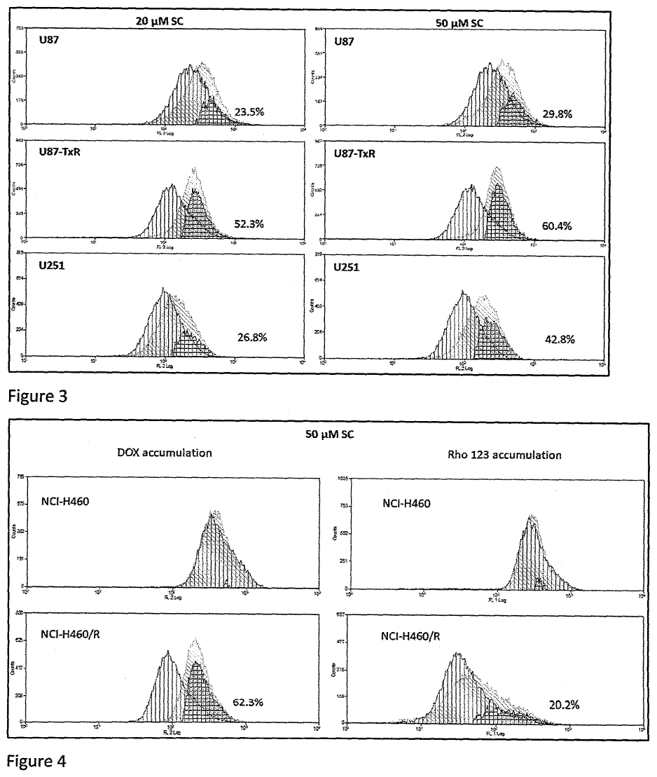

The present invention relates to cytotoxicity tests of SC on various cell types, including human glioblastoma U87, U87-TxR and U251, human non-small cell lung carcinoma NCI-H460 and NCI-H460/R, human colorectal cancer DLD1 and DLD1-TxR, and human lung fibroblasts MRC-5 (FIG. 1). Interaction studies of SC and DOX in simultaneous combined treatments on the mentioned cell lines are also included (FIG. 2). Additionally, the invention includes accumulation studies of DOX in human glioblastoma U87, U87-TxR and U251, and human non-small cell lung carcinoma NCI-H460 and NCI-H460/R (FIGS. 3 and 4), as well as a comparison of selectivity and resistant profiles of CON1 and

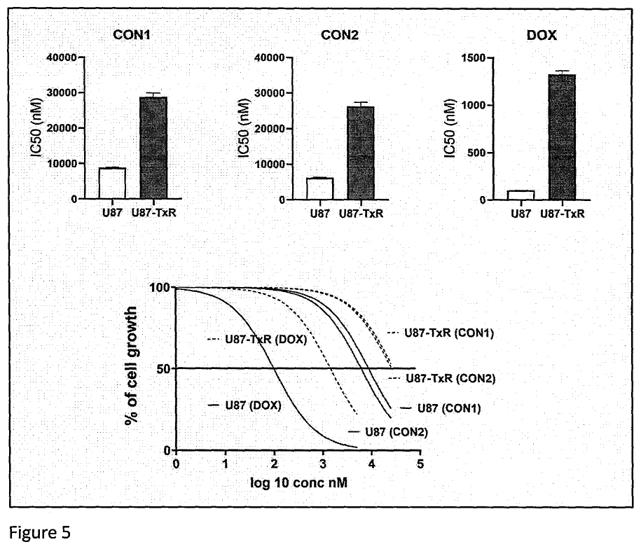

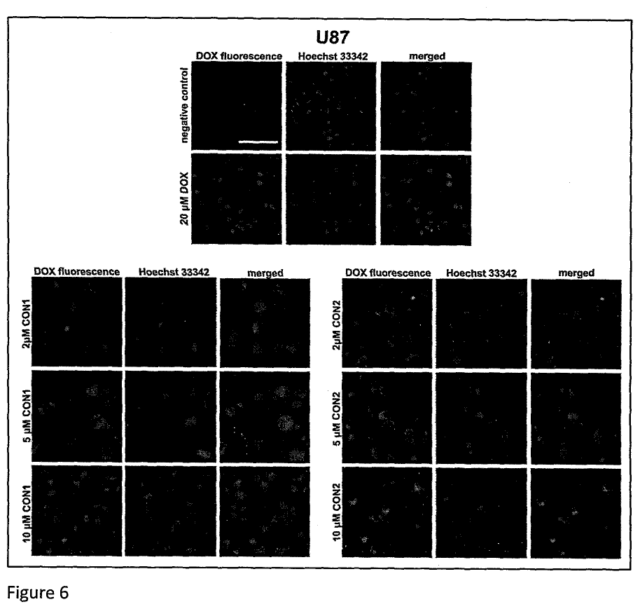

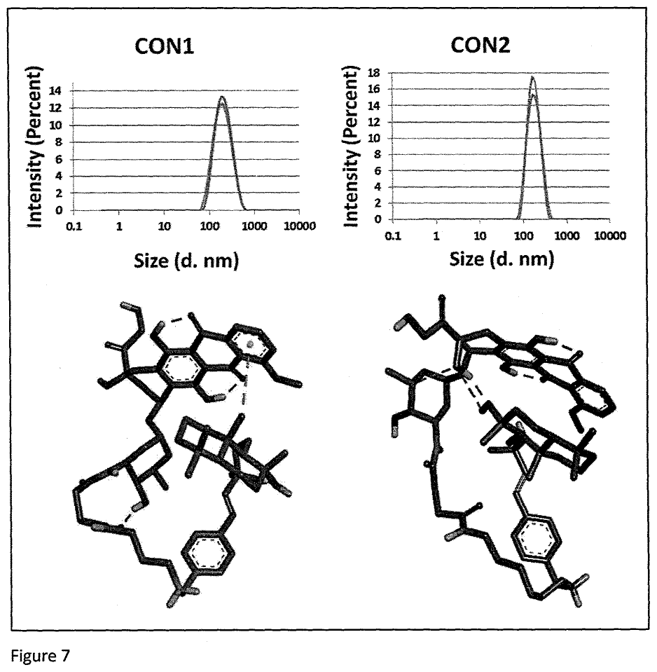

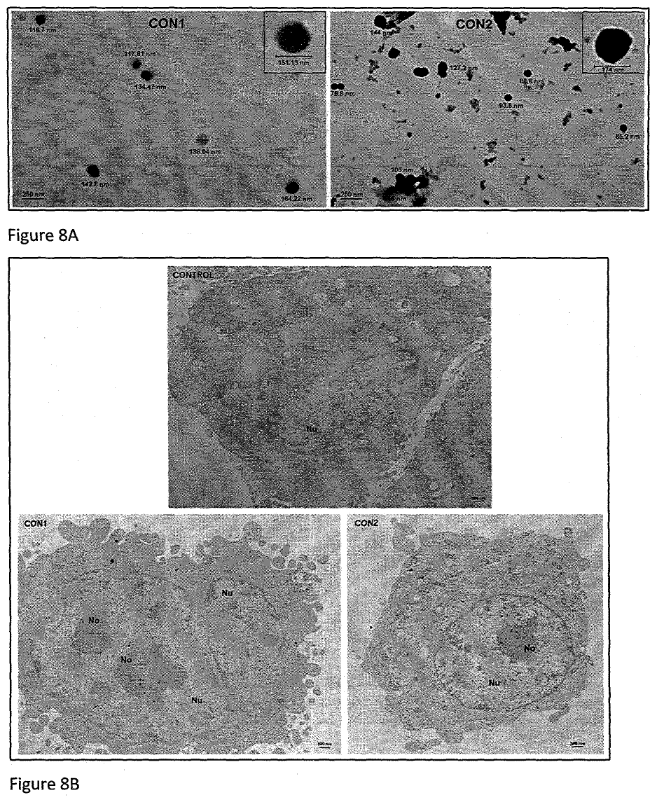

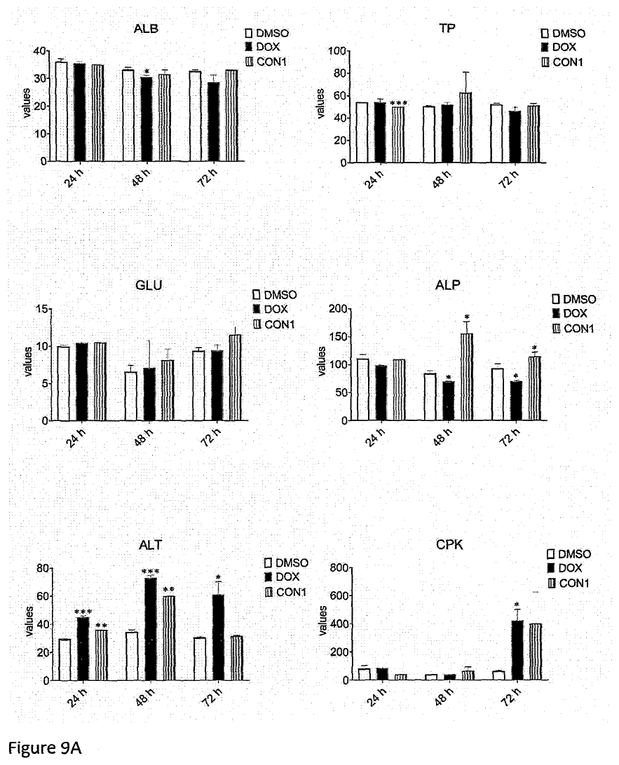

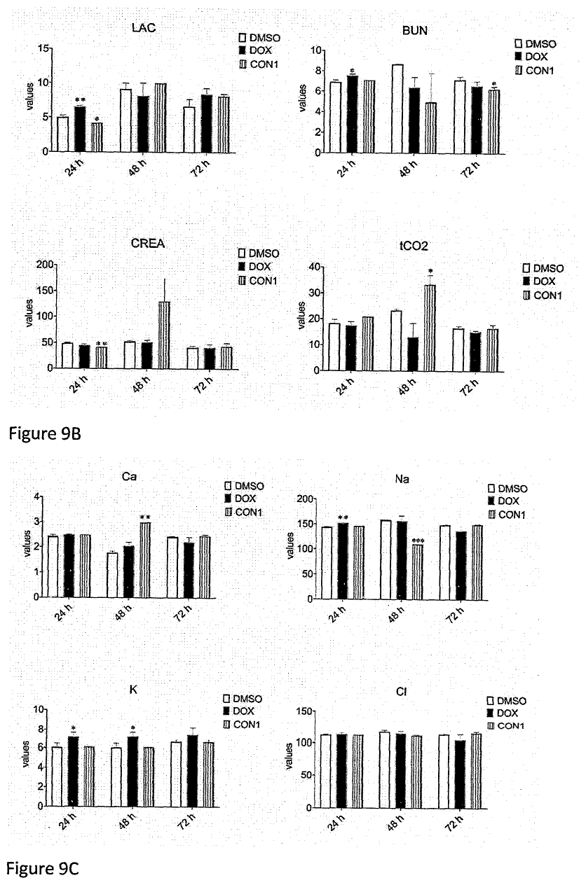

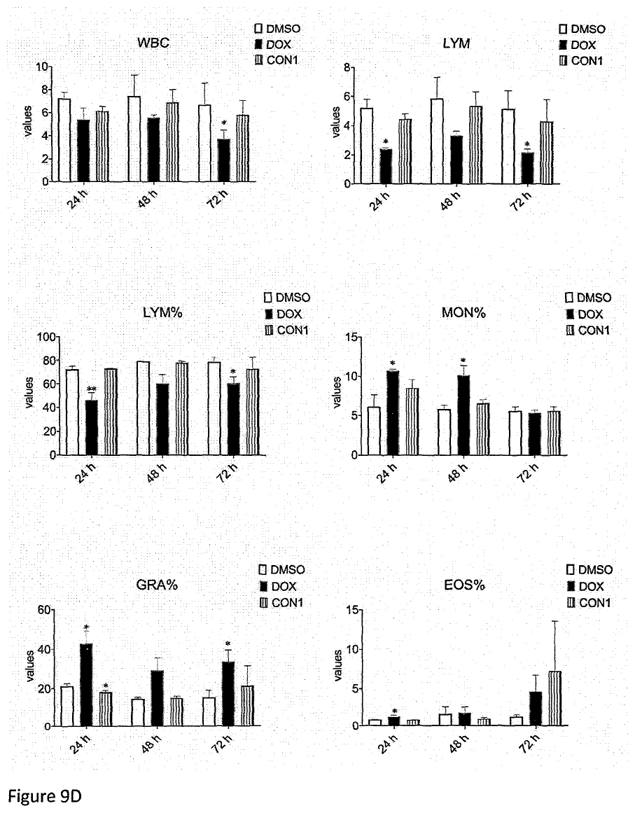

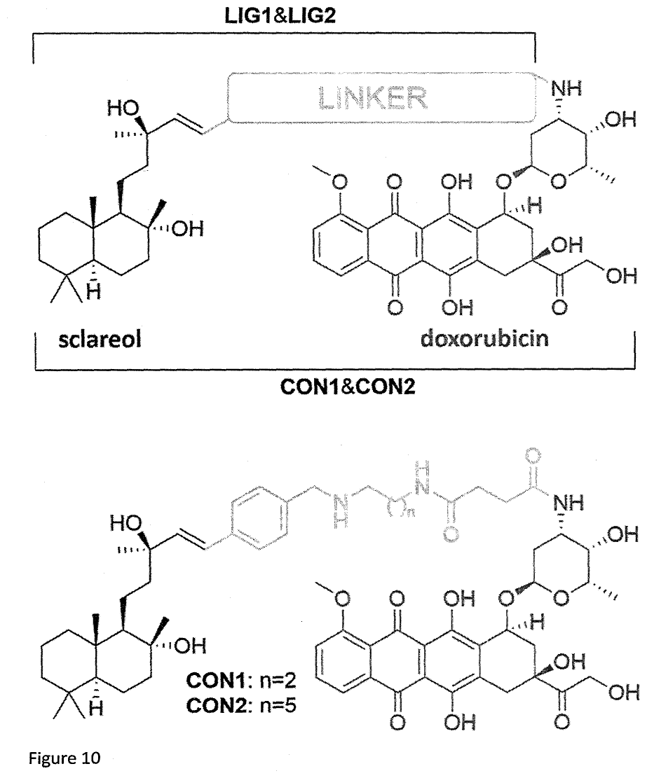

CON2 with those of DOX in glioblastoma U87 and U87-TxR, and human non-small cell lung carcinoma NCI-H460 and NCIH460/R (Table 1, FIG. 5). The invention also encompasses analyses of different intracellular localizations of CON1 and CON2 compared to DOX (FIG. 6), the unforeseen nanoparticle nature of CON1 and CON2 (Table 2, FIGS. 7 and 8), and the different toxicology profile of CON1 in comparison with DOX (FIG. 9).

SC alone has a relatively weak anticancer effect with an IC50 value of 50% inhibition of cell growth achieved in the range of 20 to 70 μM depending on the cell type, as shown in FIG. 1. In comparison, DOX acts in the nanomolar range in sensitive cancer cell lines and in the micromolar range in resistant ones, but remains below 5 M. The interaction between SC and DOX has been tested on different cell types to determine their combined treatment's nature (FIG. 2). The results showed that SC is extremely synergistic with DOX in glioblastoma cells U87-TxR and U251, as well as non-small cell lung carcinoma NCI-H460/R, which are resistant to DOX (FIG. 2). However, the combination of SC and DOX had an antagonistic effect on MRC-5 lung fibroblasts, indicating that SC reduces the effect of DOX on normal cells (FIG. 2).

It has been found that SC can increase the accumulation of DOX in glioblastoma cells, regardless of the expression and activity of P-glycoprotein. This effect was observed in all examined glioblastoma cell lines, including U87, U87-TxR and U251 (FIG. 3). Conversely, in non-small cell lung carcinoma cell lines, the accumulation increased only due to the inhibition of P-glycoprotein activity by SC in resistant NCI-H460/R cells (FIG. 4).

The newly synthesized SC and DOX conjugates were tested for cytotoxicity on different cell lines. The results showed that CON1 and CON2 were more effective in inhibiting the growth of glioblastoma cells than non-small cell lung carcinoma cells (Table 1, FIG. 5). The ligands themselves did not exhibit anticancer activity, as their IC50 values were greater than 50 μM even on sensitive cancer cell lines U87 and NCI-H460 (Table 1). The hybrid compounds were found to be selective towards cancer cells, as they had no significant effect on the growth of lung fibroblasts MRC-5 (Table 1). Furthermore, CON1 and CON2 showed better selectivity profiles than DOX in both glioblastoma and non-small cell lung carcinoma cellular models. This indicates that the selectivity index is greater for CON1 and CON2 than for DOX (Table 1). Additionally, CON1 and CON2 exhibited better resistance profiles, as the relative resistance to CON1 and CON2 was lower than that of DOX in both glioblastoma and non-small cell lung carcinoma cellular models (Table 1).

According to the fluorescence localization study, CON1 and CON2 are located near the nuclear membrane, while DOX is found inside the nucleus (FIG. 6).

Upon investigation of the physico-chemical properties of CON1 and CON2, it was discovered that these molecules have the ability to spontaneously form nanoparticles. Confirmation of this finding was obtained using Zetasizer, which revealed uniform nanoparticle sizes for both CON1 and CON2, with a low polydispersity index of less than 0.2. The positive zeta potential above 20 indicated that the formation of larger aggregates is unlikely (Table 2, FIG. 7). Furthermore, chemical modeling predicted a spontaneous, “protein-like” folding of CON1 and CON2 (FIG. 7), as a result of strong intramolecular forces. Prediction of pKa for CON1 and CON2 suggested that both molecules have protonated aliphatic nitrogen on given condition. Those structures were used for conformational search, to find stable conformational structures in solution. Most stable conformers for both molecules showed several intramolecular H-bonds, as well as additional hydrophobic interactions. Some of functional groups (—OH, amide, —NH₂+-) as well as hydrophobic structures are available for further supramolecular organization.

The formation of CON1 and CON2 nanoparticles was confirmed through transmission electron microscopy (FIG. 8A). Moreover, the transmission electron microscopy revealed that CON1 and CON2 nanoparticles can penetrate the cancer cells and can be found in various parts of the cell such as cytoplasm, cellular membranes, nucleolar membranes, mitochondrial membranes, nucleus, and nucleolus (FIG. 8B). Along with the findings obtained through fluorescent microscopy analysis, transmission electron microscopy results imply that the distribution of CON1 and CON2 compounds within the cell is distinct from that of DOX, which typically localizes in the nucleus. This also suggests that the hybrid compounds may have distinct mechanisms of action compared to DOX.

Toxicology tests conducted on Balb/c mice revealed different toxic effects of CON1 and DOX. DOX was found to significantly decrease the activity of alkaline phosphatase (ALP) and increase the levels of alanine aminotransferase (ALT) over time. This indicates that DOX impacts the liver's functioning by reducing its activity or damaging it (FIG. 9A). However, CON1 did not change ALT levels, but it did increase ALP levels after 48 h with tendency to return to control levels after 72 h (FIG. 9A). Both DOX and CON1 increased the levels of creatine phosphokinase (CPK) after 72 h. However, only DOX had a significant impact on CPK, indicating damage to the myocardium or other muscles (FIG. 9A). CON1 transiently increased total bicarbonate (tCO2) levels at 48 h, but this effect disappeared by 72 h (FIG. 9B). On the other hand, DOX consistently increased the levels of K, which implies kidney damage (FIG. 9C). CON1 showed a temporary increase in Ca levels and a decrease in Na levels at 48 h, but these changes disappeared after 72 h (FIG. 9C). The most significant toxic effect of DOX was its impact on the bone marrow, as it decreased the percentage of lymphocytes (% LYM, LYM, and white blood cells-WBC) and increased the percentage of monocytes and granulocytes (% MON and % GRA) over time. This indicates a toxic effect on the bone marrow responsible for producing these cells (FIG. 9D). Bone marrow suppression and lymphocytopenia are common side effects of DOX in cancer patients. In contrast, CON1 did not have any impact on the percentages of lymphocytes, monocytes, and granulocytes. The increase in the percentage of eosinophils (% EOS) caused by CON1 was not significantly relevant (FIG. 9D). None of the tested compounds, DOX or CON1, had any impact on the other blood parameters, such as red blood cells (RBC), hemoglobin (HGB), hematocrit (HCT), mean corpuscular volume (MCV), mean corpuscular hemoglobin (MCH), mean corpuscular hemoglobin concentration (MCHC), red blood cell distribution width (RDW), platelets (PLT), or mean platelet volume (MPV). This data was not shown.

TABLE 1
Anticancer effects of DOX, SC, CON1, CON2, LIG1, and LIG2 expressed in IC50* values

| | | U87- | Relative | | Selectivity | NCI- | NCI- | Relative | | Selectivity |
|---|---|---|---|---|---|---|---|---|---|---|
| | U87 | TxR² | Resistance³ | MRC-5⁴ | Index⁵ | H460 | H460/R² | Resistance³ | MRC-5⁴ | Index⁵ |
| DOX¹ | 100.1 | 1323.9 | 13.2 | 294.6 | 2.9 | 127.5 | 2496 | 19.6 | 588 | 4.6 |
| SC | 51.6 | 39.2 | 0.8 | 75.9 | 1.5 | 40.3 | 47.4 | 1.2 | 64.1 | 1.6 |
| CON1 | 8.8 | 28.7 | 3.3 | 137.2 | 15.6 | 9.8 | 38.4 | 3.9 | 72.6 | 7.4 |
| CON2 | 6.2 | 26.2 | 4.2 | >400 | >64.5 | 25.1 | 83.3 | 3.3 | 131.4 | 5.2 |
| LIG1 | 74.4 | 61.7 | 0.8 | 236.0 | 3.2 | 41.7 | 55.2 | 1.3 | 392.6 | 9.4 |
| LIG2 | 344.6 | 273.1 | 0.8 | >400 | >1.2 | 99.6 | 109.8 | 1.1 | 610.1 | 6.1 |

*The IC50 value is the concentration that causes 50% inhibition of cell growth.
¹IC50 values for DOX are in nanomoles, and for other compounds in micromoles.
²Multidrug resistant (MDR) cancer cell lines obtained by continuous exposure to antitumor drugs (paclitaxel in the case of U87-TxR, and doxorubicin in the case of NCI-H460/R).
³The relative resistance obtained as the ratio between the IC50 value for the MDR cancer cell line and the corresponding sensitive cell line.
⁴In experiments with glioblastoma cells, MRC-5 cells were initially seeded at a density of 4,000 cells per well, and in experiments with non-small cell carcinoma cells at a density of 2,000 cells per well, so that their density is the same as the density of the cancer cells with which they were compared.
⁵Selectivity index obtained as a ratio between the IC50 value for MRC-5 and the corresponding sensitive cell line.

TABLE 2
Nanoparticle nature of CON1 and CON2 studied by Zetasizer

| | Z-Average | Polydispersity index | Zeta Potential |
|---|---|---|---|
| | (d · nm) | (PdI) < 0.2 | (mV) > 20 |
| CON1 | 189.4 ± 9.7 | 0.198 ± 0.038 | 39.6 ± 4.3 |
| CON2 | 180 ± 14.7 | 0.103 ± 0.016 | 40.2 ± 1.0 |

The results indicate that SC and DOX hybrid compounds CON1 and CON2 (FIG. 10) are more effective in terms of their anticancer properties compared to DOX.

The results indicate that SC and DOX hybrid compounds CON1 and CON2 are more effective in terms of their anticancer properties compared to DOX. They are taken up by cancer cells, the level of resistance is lower, the selectivity towards cancer cells is higher, they have nanoparticle nature and different intracellular distribution, and better toxicology profile than DOX. The study shows that by conjugating SC and DOX, the anticancer properties of DOX can be improved, reducing resistance and increasing the selectivity towards cancer cells.

The current invention relates to a couple of novel hybrid compounds that are formed by the hybridization of SC and DOX named CON 1 and CON 2. These compounds have displayed excellent anticancer properties against various cell lines, including glioblastoma, non-small cell lung carcinoma, and colorectal carcinoma. The hybrid compounds have a higher level of selectivity towards cancer cells than DOX, and they also show lower levels of resistance in resistant cancer cells. Furthermore, the localization of CON1 and CON2 inside cancer cells helps prevent extensive double-stranded DNA damage caused by DOX binding to DNA chains. Unforeseen nanoparticle nature of CON1 and CON2 and CON1's lower toxicity profile in mice compared to DOX further enhance their medical value.

The hybrid compounds of SC and DOX can be used as medicine in medical products and pharmaceutical preparations for the treatment of various types of cancer. These compounds can be formulated into various dosage forms, such as injectable solutions, liposomal formulations, and solid dosage forms, depending on the specific requirements of the patient. The compounds can also be used in combination with other chemotherapy drugs or radiation therapy to enhance their effectiveness in treating cancer.

The present invention demonstrates an unforeseen ability to inhibit cancer growth in various cell lines, including human glioblastoma, non-small cell lung carcinoma, and colorectal carcinoma. Consequently, it finds the usage as a medicine in both medical products and pharmaceutical preparations.

The present invention focuses on sclareol conjugate derivatives with doxorubicin, their pharmaceutical salts, synthesis, and application in cancer treatment. These compounds have been found to possess antitumor and antiproliferative activity on cancer cells, particularly on human glioblastoma, non-small cell lung carcinoma, and colorectal carcinoma.

### Preparations

The invention of sclareol conjugate derivatives with doxorubicin and their pharmaceutical salts has brought about a revolutionary change in treating cancer. These compounds are synthesized to exhibit in vitro anticancer and antiproliferative activity on cancer cells, specifically on human glioblastoma, non-small cell lung carcinoma, and colorectal carcinoma.

The main objective of this invention is to provide novel compounds that can be used as active agents in cancer therapy. The compounds are designed to treat and/or prevent proliferative and/or neoplastic diseases. They are also developed to inhibit growth of cancer cells, especially in humans. Pharmaceutical compositions/preparations comprising the compound conjugates of sclareol with doxorubicin and a pharmaceutically acceptable carrier, excipient can now be used as medicine in treating cancer, specifically human glioblastoma, non-small cell lung carcinoma, and colorectal carcinoma. These conjugates offer a new hope for cancer patients by providing an effective treatment option.

Moreover, the invention provides conjugates of sclareol with doxorubicin for use in the manufacture of a medicament for the treatment of cancer in mammals. The compounds are specifically designed to kill cancer cells and/or inhibit cancer cell replication in mammals, especially humans.

### INDUSTRIAL APPLICABILITY

The invention describes a three-step procedure for producing a conjugate via convergent synthesis. The conjugates obtained through this method have been found to possess favorable anticancer characteristics, exhibiting a higher selectivity towards cancer cells compared to the starting compounds sclareol and doxorubicin. Additionally, they exhibit a lower degree of resistance compared to doxorubicin. These compounds can be used for the preparation of medicinal products, specifically for the treatment of cancer, such as glioblastoma, non-small cell lung carcinoma, and colorectal carcinoma.

The invention provides medicinal products comprising a sclareol derivative and doxorubicin or a pharmaceutically acceptable salt thereof, along with a pharmaceutically acceptable carrier. These medical products are used in therapy for cancer treatment.

The pharmaceutical preparations/compositions of the invention comprises an effective dose of the conjugate of sclareol and doxorubicin in the form of a pharmaceutically acceptable salt, solvate, or hydrate, along with at least one pharmaceutically acceptable excipient. The excipients are selected according to the desired mode of administration and the pharmaceutical form.

The mentioned pharmaceutical forms can be administered orally, sublingually, subcutaneously, intramuscularly, intravenously, topically, locally, intratracheally, intranasally, transdermally, rectally, or intraocularly. The main active ingredient conjugate of sclareol and doxorubicin can be administered as a single form of administration or as a mixture with at least one pharmaceutical excipient.

The pharmaceutical forms of administration can be in the form of tablets, gel capsules, granules, powders, oral or injectable solutions or suspensions, transdermal patches, forms for administration sublingually, buccally, intratracheally, intraocularly, intranasally or by inhalation, topically, transdermally, subcutaneously, intramuscularly, intravenously, forms of rectal administration, or implants. For the topical administration, creams, gels, ointments, lotions, or eye drops may be considered.

These pharmaceutical forms are prepared using known conventional methods.

1. Hybrid compounds CON1 and CON2 conjugates of sclareol and doxorubicin of formula: 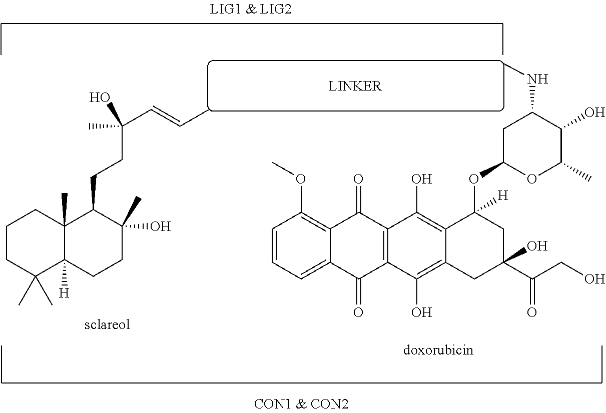
and/or their pharmaceutically acceptable salts or hydrates or solvates
wherein the ligands LIG1 and LIG2 are: 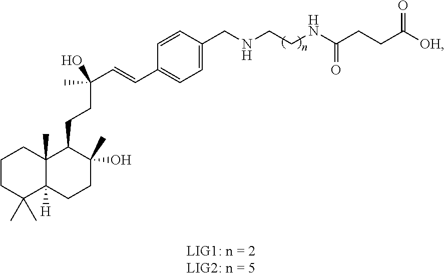
CON1 and CON2 compounds are:

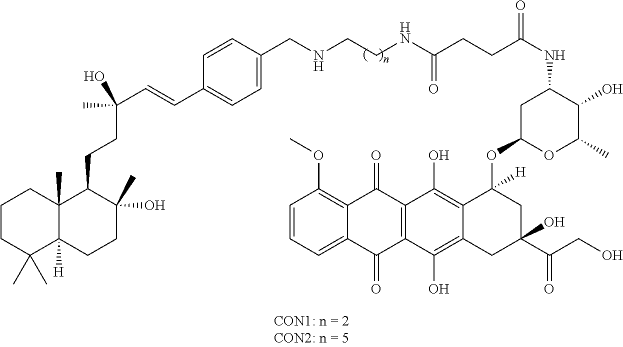

2. Compounds of claim 1, wherein CON1 is N1-((25,35,4S,6R)-3-hydroxy-2-methyl-6-(((1S,3S)-3,5,12-trihydroxy-3-(2-hydroxyacetyl)-10-methoxy-6,11-dioxo-1,2,3,4,6,11-hexahydrotetracen-1-yl)oxy)tetrahydro-2H-pyran-4-yl)-N4-(3-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)propyl) succinamide; and CON2 is N1-((2S,3S,4S,6R)-3-hydroxy-2-methyl-6-(((15,3S)-3,5,12-trihydroxy-3-(2-hydroxyacetyl)-10-methoxy-6,11-dioxo-1,2,3,4,6,11-hexahydrotetracen-1-yl)oxy)tetrahydro-2H-pyran-4-yl)-N4-(6-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)hexyl) succinamide, and pharmaceutically acceptable salts, hydrates and/or solvates thereof.

3. Hybrid compounds CON1 and CON2 conjugates of claims 1 to 2, wherein doxorubicin and sclareol are covalently linked via a linker in a molar ratio of 1:1.

4. The method for producing hybrid compounds of sclareol and doxorubicin (conjugates) CON1 and CON2, of formula 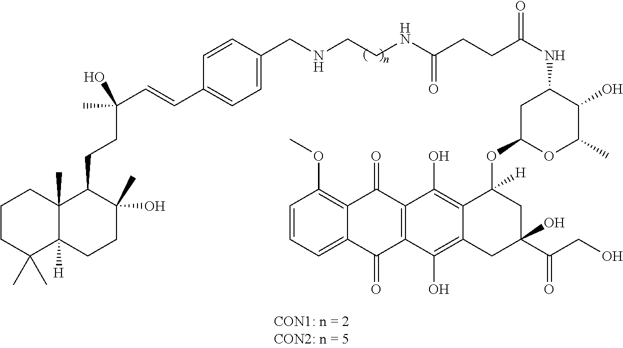
comprises the following reaction steps:
a) reaction of sclareol with 4-formylphenylboronic acid in the presence of a palladium-based catalyst to yield the corresponding derivative 1. 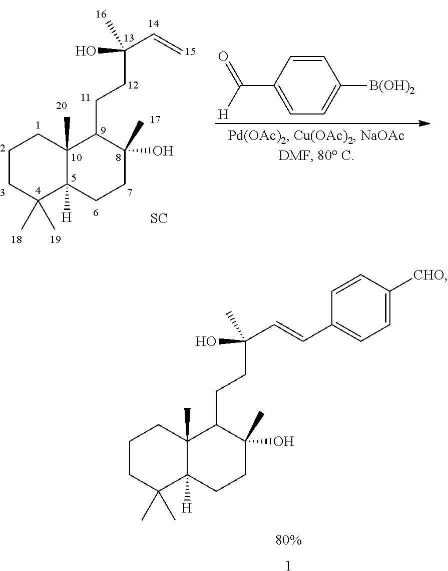
b) reaction of alkyl diamines, 1,3-diaminopropane, and 1,6-diaminohexane, and succinic anhydride, synthesizing compounds 2 and 3. 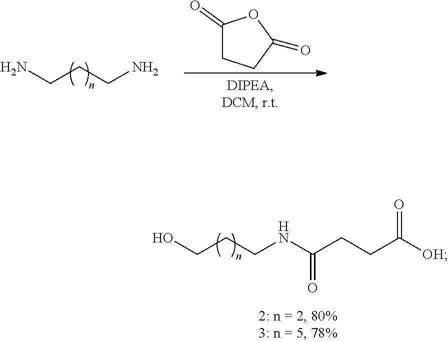
c) reaction of sclareol derivative 1 and compounds 2 and 3 by adding sodium borohydride in the presence of titanium (IV) isopropoxide, resulting in the formation of corresponding ligands LIG1 and LIG2. 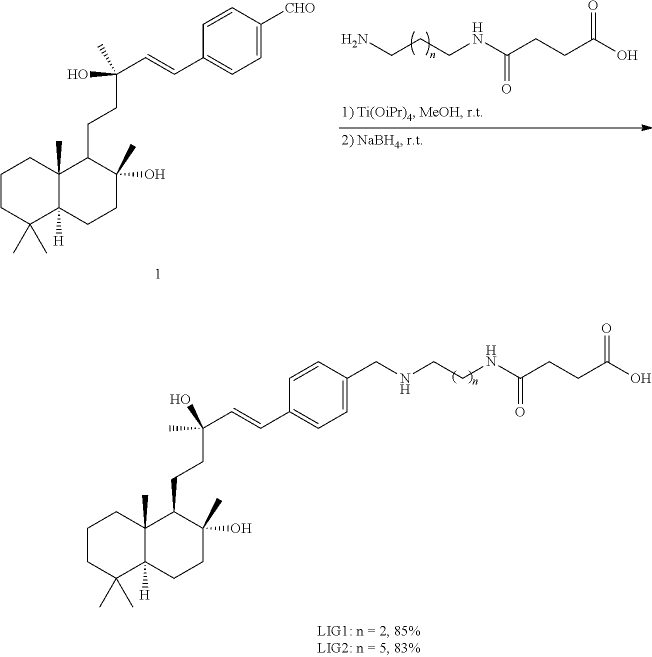
d) an amide bond link formation between doxorubicin and ligands LIG1 and LIG2, specifically by adding 1-hydroxybenzotriazole and 1-ethyl-3-(3-dimethylaminopropyl) carbodiimide, yielding conjugates CON1 and CON2

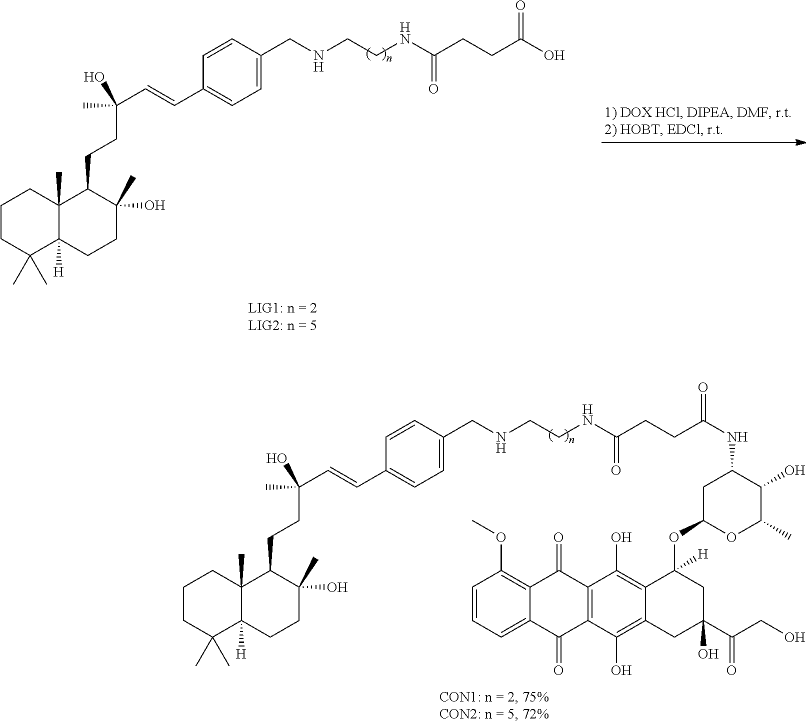

5. Ligand compounds LIG1 and LIG2 of formula: 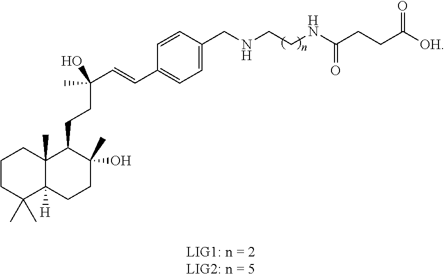
produced by the method of claim 4, steps a)-c).

6. Compounds of claim 5, wherein LIG1 is 4-((3-((4-((R,E)-3-hydroxy-5-((1R,2R,4aS,8aS)-2-hydroxy-2,5,5,8a-tetramethyldecahydronaphthalen-1-yl)-3-methylpent-1-en-1-yl)benzyl)amino)propyl)amino)-4-oxobutanoic acid, and LIG2 is 4-((6-((4-((R,E)-3-hydroxy-5-((1R,2R,4aS,8aS)-2-hydroxy-2,5,5,8a-tetramethyldecahydronaphthalene-1-yl)-3-methylpent-1-en-1-yl)benzyl)amino)hexyl)amino)-4-oxobutanoic acid.

7. The method for producing the compounds LIG1 and LIG2, of claim 6, comprising steps a)-c) of claim 4.

8. A composition comprising one hybrid compound according to claims 1 to 3, or pharmaceutically acceptable salt, hydrate or solvate thereof optionally combined with at least one pharmaceutically acceptable excipient.

9. Hybrid compounds of claims 1 to 3, or pharmaceutically acceptable salts or hydrates or solvates thereof, or a composition of claim 8 for use as a medicament.

10. The hybrid compounds of claims 1 to 3, or pharmaceutically acceptable salts, hydrates, or solvates thereof, or a composition of claim 8 for use in treating cancer in humans, including but not limited, to non-small cell lung carcinoma, glioblastoma, and colorectal carcinoma.

11. A method for producing sclareol derivative 1 that includes step a) of claim 4.

12. Sclareol derivative 1 produced according to claim 11, characterized in that, it is 4-{(1E,3R)-3-hydroxy-5-[(2R,4aS,8aS)-2-hydroxy-2,5,5,8a-tetramethyldecahydronaphthalen-1-yl]-3-methylpent-1-en-1-yl}benzaldehyde.
